(19) United States
(12) Patent Application Publication
(10) Pub. No.: US 2024/0197243 A1
(43) Pub. Date: Jun. 20, 2024

(54) FRAGRANCE FOR IMPROVING RELAXATION STATE AND METHOD OF ASSESSING

(71) Applicant: GIVAUDAN SA, VERNIER (CH)

(72) Inventors: Giuliano GAETA, Ramsgate Kent (GB); Alan Forbes PROVAN, Kingsnorth, Ashford Kent (GB); Natalie Anuradha T.D. GUNASEKARA, Pinner, Greater London (GB); Ioannis KONTARIS, Ashford Kent (GB)

(21) Appl. No.: 18/555,705

(22) PCT Filed: May 30, 2022
(86) PCT No.: PCT/EP2022/064552

§ 371 Date: Oct. 16, 2023

(30) Foreign Application Priority Data
May 28, 2021 (GB) 2107716.9

(51) Int. Cl. A61B 5/00; A61B 5/16; A61M 21/00; A61M 21/02; C11B 9/00

(52) U.S. Cl. CPC A61B 5/4836; A61B 5/0075; A61B 5/165; A61M 21/02; C11B 9/0061; A61M 2021/0016

(57) ABSTRACT

The present disclosure relates to methods of assessing the ability of a test fragrance ingredient or a test fragrance composition to improve the relaxation state of a human subject and of creating fragrance compositions having a relaxing effect on a human subject. It further relates to fragrance compositions for improving the relaxation state of a human subject, to consumer products comprising such fragrance compositions, and to methods of improving the relaxation state of a human subject.

The present invention relates to methods of assessing the ability of a test fragrance ingredient or a test fragrance composition to improve the relaxation state of a human subject and of creating fragrance compositions having a relaxing effect on a human subject. It further relates to fragrance compositions for improving the relaxation state of a human subject, to consumer products comprising such fragrance compositions, and to methods of improving the relaxation state of a human subject.

Perfumery has been widely employed by consumer product companies in order to impart to their products pleasant, well-liked odours that promote consumer liking and which influence purchasing decisions for this reason.

However, in an increasingly competitive marketplace, mere liking is often not sufficient to differentiate one brand over its competitors. Accordingly, in the market execution of their products, consumer product companies frequently refer to wide-ranging product benefits, typically communicated through diverse advertising campaigns, as well as on the packaging and labelling of their products, which together form an important part of their branding strategy. New differentiating effects are constantly sought, and perfumery has often been employed as a means to achieve those effects. For example, perfumery has been employed to create real or perceived functional effects that may relate to cosmetic effects, hygiene effects, malodour-counteracting effects, and the like.

It has long been known that fragrance materials and essential oils can promote feelings of relaxation and well-being. These materials have also been used in cosmetic products and aromatherapy in order to provide a similar effect.

Aroma-Chology® is a term coined by the Olfactory Research Fund Ltd. (see extensive review by J. Jellinek in Cosmetics & Toiletries, (1994) 109, pp 83-101). It is concerned specifically with the temporary, beneficial psychological effects of aromas in human behaviours and emotions to improve mood and quality of life. In fact, a large number of products promoted as having aromatherapy benefits can be more accurately identified for their Aroma-Chology® benefits as they product temporary psychological effects. However, there is no teaching as to how to formulate products to achieve such benefits qualitatively or quantitatively with a reliable expectation of success. In addition, it is well known that fragrances can be perceived as associated with different attributes in different countries.

More recently, several patent applications (e.g. WO 02/49600, WO 2008/050084, WO 2008/050086, WO 2020/165463) have focused on providing positive mood benefits through fragrance compositions, providing guidelines on how to measure these mood benefits and on how to create effective fragrance compositions.

For instance, WO 02/49600 describes fragrance compositions, which aim to induce or be associated with positive, low activation moods and emotions. These compositions may be used to deliver positive mood benefits, and in particular relaxation, to a human subject. WO 02/49600 provides formulation guidelines specifying different types of fragrance ingredients (e.g. relaxing, non-relaxing or neutral ingredients) and the concentrations at which these fragrance ingredients may be present in the compositions in order to achieve the desired effect. These formulation guidelines have been developed based on consumer testing and EEG (electroencephalography) measurements.

WO 02/49600 classifies fragrance ingredients into three categories: relaxing fragrance ingredients (R) that induce positive, low activation moods and emotions, such as relaxation (i.e. relaxing properties); non-relaxing fragrance ingredients (NR) that induce negative, high activation moods or emotions (i.e. non-relaxing properties); and neutral fragrance ingredients (N) having a neutral effect in terms of relaxing properties.

However, there is a large group of other fragrance ingredients that have not been assigned to any of these three groups. Also, some fragrance ingredients are now longer used nowadays, either due to regulatory restrictions or due to the availability of better alternatives.

There is therefore a need to classify further fragrance ingredients.

WO 02/49600 uses EEG for assessing the influence of a fragrance composition on the mood and emotions of test subjects. However, EEG has several important drawbacks:
low spatial resolution
lack of robustness to motion (severely prone to artefacts associated to muscle activity, e.g. eye blinking)
lack of information on cerebral blood flow
higher difficulty to obtain clean signals before starting data collection

Most importantly, subjects should move as little as possible during an EEG measurement in order to avoid interference. This makes an accurate in-context-testing essentially impossible.

It is therefore highly desirable that an improved technique for assessing the mood state of a human subject is developed, which allows for more flexibility during the measurement.

The above problems are solved by the present invention.

In a first aspect, the present invention provides a method of assessing the relaxation state of a human subject by means of fNIRS (functional near-infrared spectroscopy).

In a second aspect, the present invention provides a method of assessing the ability of a test fragrance ingredient or a test fragrance composition to improve the relaxation state of a human subject.

In a third aspect, the present invention provides a method of creating a fragrance composition having a relaxing effect on a human subject.

In a fourth aspect, the present invention provides a fragrance composition for improving the relaxation state of a human subject.

In a fifth aspect, the present invention provides a consumer product comprising said fragrance composition.

In a sixth aspect, the present invention provides a method of improving the relaxation state of a human subject, comprising the step of providing an effective amount of the fragrance composition of the invention to the human subject.

In a seventh aspect, the present invention relates to the use of certain fragrance ingredients for improving the relaxation state of a human subject.

The use of functional near-infrared spectroscopy (fNIRS) for assessing the mood state, and in particular the relaxation state, of a human subject is highly advantageous: fNIRS is harmless, tolerant to bodily movements, and highly portable; it is also suitable for all possible participant populations, from newborns to the elderly, and experimental settings, both inside and outside the laboratory (for a review, see: “The present and future use of functional near-infrared spectroscopy (fNIRS) for cognitive neuroscience”, Pinti et al., Ann. N.Y. Acad. Sci. 1464 (2020) 5-29). In particular, the use of fNIRS allows for in-context-testing, where participants are asked to perform specific tasks related to the fragrances provided to them (e.g. cleaning a hard surface while smelling the fragrance of an all-purpose cleaner).

fNIRS is an optical, non-invasive neuroimaging technique that allows the measurement of brain tissue concentration changes of oxygenated (Oxy Hb or HbO₂) and deoxygenated (Deoxy Hb or HbR) haemoglobin following neuronal activation. This is achieved by shining NIR light (650-950 nm) into the head, and, taking advantage of the relative transparency of the biological tissue within this NIR optical window, light will reach the brain tissue. The most dominant and physiological-dependent absorbing chromophore within the NIR optical window is haemoglobin. Based on its saturation state, we can have haemoglobin in its oxygenated (HbO₂) and deoxygenated form (HbR). In particular, HbO₂ and HbR absorb the NIR light differently: HbO₂ absorption is higher for λ>800 nm; on the contrary, HbR absorption coefficient is higher for λ<800 nm.

When a brain area is active and involved in the execution of a certain task, the brain's metabolic demand for oxygen and glucose increases, leading to an oversupply in regional cerebral blood flow (CBF) to meet the increased metabolic demand of the brain. The oversupply in regional CBF produces an increase in HbO₂ and a decrease in HbR concentrations; these are estimated by changes in light attenuation that can be measured by fNIRS.

The portion of tissue interrogated by the NIR light is called a channel and is located at the midpoint between the source optode (S) and the detector optode (D), and at a depth of around the half of the source-detector separation. To fully exploit the potential of fNIRS, multi-channel devices are used nowadays. These allow monitoring of larger portions of the head and the gathering of topographic HbO₂ and HbR maps. Several multi-channel fNIRS devices are commercially available (e.g. Brite by Artinis, ETG-4100 by Hitachi or NIRSPort by NIRx).

The position of the fNIRS channels is generally standardized based on the EEG's 10-20 system. Typical devices use about sixteen to twenty-two channels. Optodes (detectors and sources) must be placed in an alternate fashion (i.e. a source followed by a detector, followed by a source . . . ) typically in a grid with equal distances between the channels, e.g. at a distance of 3 cm (Pinti et al. (2019) “Current Status and Issues Regarding Pre-processing of fNIRS Neuroimaging Data: An Investigation of Diverse Signal Filtering Methods Within a General Linear Model Framework”, Front. Hum. Neurosci. 12:505). Both optodes and channels are typically numbered to allow for identification. For optodes, the letter S before the number typically defines a source optode, while the D letter before the number defines a detector optode. The numbers are usually progressive, e.g. from 1 to 8 for the sources and from 1 to 7 for the detectors.

In the methods of the present invention, the following set-up of the fNIRS channels was used:

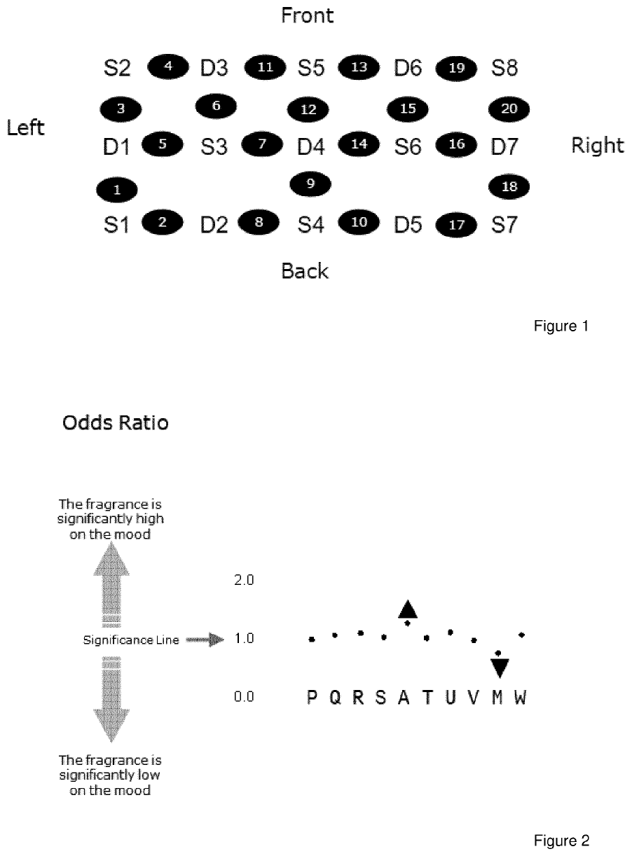

The centre of fNIRS Channel 12 was placed on the standard position EEG channel FPz according to the EEG 10-20 system (Trambaiolli et al. “Predicting affective valence using cortical hemodynamic signals”, Sci Rep 8, 5406 (2018)). Channel 12 is located between source S5 and detector D4, where S5 is situated 1.5 cm from the location of the EEG channel FPz towards the Nasion on the midline of the head, and D4 is situated 1.5 cm from the location of EEG channel FPz towards the Inion on the midline of the head. All fNIRS optodes are placed at a standardised distance of 3 cm one from one another and are arranged on gridlines extending parallel and orthogonally to the midline. Taking S5 and D4 as a reference, and considering a shift of 3 cm for each optode either on the Nasion-Inion direction (where “in front” means towards the Nasion and “behind” means towards the Inion) or on the Pre Auricolar line (where “to the left” means towards the Left Pre Auricolar line and “to the right” means towards the Right Pre Auricolar Line), then S4 is behind D4, D5 on the right of S4, S7 on the right of D5, D7 in front of S7, S8 in front of D7, S6 on the left of D7, D6 on the left of S8, D2 on the left of S4, S3 on the left of D4, D3 in front of S3, S1 on the left of D2, D1 in front of S1 and S2 in front of D1. This setup is also shown in FIG. 1.

The channel scheme is the following:

| Channel | Position |
|---|---|
| Number | (Source - Detector) |
| Channel 1 | S1 - D1 |
| Channel 2 | S1 - D2 |
| Channel 3 | S2 - D1 |
| Channel 4 | S2 - D3 |
| Channel 5 | S3 - D1 |
| Channel 6 | S3 - D3 |
| Channel 7 | S3 - D4 |
| Channel 8 | S4 - D2 |
| Channel 9 | S4 - D4 |
| Channel 10 | S4 - D5 |
| Channel 11 | S5 - D3 |
| Channel 12 | S5 - D4 |
| Channel 13 | S5 - D6 |
| Channel 14 | S6 - D4 |
| Channel 15 | S6 - D6 |
| Channel 16 | S6 - D7 |
| Channel 17 | S7 - D5 |
| Channel 18 | S7 - D7 |
| Channel 19 | S8 - D6 |
| Channel 20 | S8 - D7 |

Thus, there are nine channels per hemisphere (left or right) and two channels at the midline of the frontal and prefrontal areas. Channels 1 to 8 and 11 are located in the left hemisphere, Channels 9 and 12 are on the midline, and Channels 10 and 13 to 20 are in the right hemisphere. Channels 9 and 12 are only considered for full brain analysis.

By means of extensive research, it has been found that certain areas of the brain, and in particular certain channels, can be used as indicators for assessing the relaxation state of a human subject. More specifically, an increase or decrease of Oxy Hb, Deoxy Hb and/or Total Hb (corresponding to the sum of Oxy Hb plus Deoxy Hb) in the left or right hemisphere, the full brain, or certain specific channels, at certain time points provides an indication as to whether the relaxation state of the human subject is increased or decreased or stays about the same. The details will be described in relation to the method outlined below, but equally apply to the general method of assessing the relaxation state of a human subject.

The above finding has been applied in the present invention to provide a method of assessing the ability of a test fragrance ingredient or a test fragrance composition to improve the relaxation state of a human subject.

Said method comprises the following steps:
a) measuring a base relaxation state of one or more human test subject(s);
b) providing the test fragrance ingredient or the test fragrance composition to the human test subject(s) for smelling;
c) measuring a resulting relaxation state of the human test subject(s); and
d) determining a difference between the resulting relaxation state and the base relaxation state for the human test subject(s).

The base relaxation state and the resulting relaxation state are measured by functional Near Infrared Spectroscopy (fNIRS) of the human test subject(s)' left brain hemisphere, right brain hemisphere, and full brain.

In order to measure the base relaxation state, the human test subject(s) may be provided with a non-fragranced sample, e.g. a piece of cotton or cloth or a sorbarod. A sorbarod is a small plastic pot containing a polyester absorbent fibre insert encased in polyethylene sleeve. The fragrance can be applied to the insert, which provides continuous refreshment of the fragrance over several assessments, and can be easily perceived when held close to the nose to smell.

Alternatively, it is also possible to measure a reference relaxation state, e.g. in the presence of a reference fragrance sample.

If more than one human test subjects are involved, results for the base relaxation state and the resulting relaxation state may be averaged prior to determining the difference. Alternatively, it is also possible to determine the difference for each human test subject separately.

It has been found that the test fragrance ingredient or the test fragrance composition is able to improve the relaxation state of the human subject if at least three out of the following six conditions are met:
Condition 1: Deoxy Hb for the right brain hemisphere shows a statistically significant increase after 5-10 seconds of smelling;
Condition 2: Oxy Hb for the full brain shows a statistically significant decrease after 30 seconds of smelling;
Condition 3: Oxy Hb for the right brain hemisphere shows a statistically significant decrease after 0-10 seconds of smelling;
Condition 4: Oxy Hb for the right brain hemisphere shows a statistically significant decrease after 0-5 seconds of smelling;
Condition 5: Oxy Hb for the full brain shows a statistically significant decrease after 0-5 seconds of smelling;
Condition 6: Oxy Hb for the right brain hemisphere shows a statistically significant decrease after 30 seconds of smelling.

As outlined above, Total Hb is the amount of total haemoglobin measured, Oxy Hb is the amount of oxygenated haemoglobin measured, and Deoxy Hb is the amount of deoxygenated haemoglobin measured.

Haemoglobin values for the left brain hemisphere correspond to the mathematical average of the individual haemoglobin values of Channels 1 to 8 and 11, as defined above.

Haemoglobin values for the right brain hemisphere correspond to the mathematical average of the individual haemoglobin values of Channels 10 and 13 to 20, as defined above.

Haemoglobin values for the full brain correspond to the mathematical average of the individual haemoglobin values of all Channels 1 to 20, as defined above.

The effect on haemoglobin levels (Total Hb, Oxy Hb, and Deoxy Hb) may vary over time. It was found that more accurate results can be obtained by analysing haemoglobin values for several different time periods, e.g. after 0-5 seconds, 0-10 seconds, 5-10 seconds, 10-15 seconds, 15-20 seconds, 10-20 seconds, 20-25 seconds, 25-30 seconds, or after 30 seconds. Interestingly, the 5-second blocks roughly correspond to the time of a full respiration cycle (inhalation+exhalation).

Statistical significance is verified using a 2-tailed Student's t-test with a statistical significance threshold at 0.05.

Throughout this application, the terms “improving the relaxation state”, “increasing the relaxation state”, and “enhancing the relaxation state” are used interchangeably. They are meant to express that a certain item, in particular a fragrance ingredient or fragrance composition or consumer product containing the same, has a relaxing effect on a human subject. In other words, they induce positive, low activation moods and emotions, such as relaxation (i.e. they have relaxing properties).

This emotional territory has been defined typically by the model circumplex of affect (Posner J, Russell J A, Peterson B S. The circumplex model of affect: an integrative approach to affective neuroscience, cognitive development, and psychopathology. Dev Psychopathol. 2005; 17(3): 715-734. doi:10.1017/S0954579405050340), with the positive low activated moods represented by feelings of calm, relaxation and serenity. This emotional space has been found to further include positive deactivated emotions, such as de-stressed, contemplative, mindful, and balanced, to reflect those inner feelings of relaxation, and also positive calming feelings from others in terms of empathy, cared for and loving.

Thus, the present application in general relates to the enhancement of positive low activated moods, positive deactivated emotions, inner feelings of relaxation, and positive calming feelings from others, including but not limited to, feelings of calm, relaxation, serenity, de-stressed, contemplative, mindful, balanced, empathy, cared for and loving.

Throughout this application, the terms “fragrance” and “perfume” are used interchangeably.

Furthermore, also the terms “(fragrance) ingredient” and “(fragrance) material” are used interchangeably. In the context of the present invention, the term “fragrance ingredient” refers to an ingredient that has the function of providing a noticeable and identifiable odour to the fragrance composition. Fragrance ingredients include highly performing ingredients intended for providing an intense olfactive impression, as well as less performing ingredients intended for providing a subtle olfactive impression.

The term “fragrance composition” relates to a mixture of two or more fragrance ingredients. It may optionally include one or more odourless or low-odour solvents and/or diluents, e.g. as a vehicle for a fragrance material.

Throughout this application, the terms “(human) test subjects” and “participants” are used interchangeably.

Preferably, several human test subjects are involved in the method of the invention, in order to get a more representative and reliable result, for example more than five, more than ten, more than 15, or even more. Results from several human test subjects may be averaged. Alternatively, they may also be summed up.

Furthermore, participants that indicate that they dislike a certain test fragrance ingredient or test fragrance composition may be excluded from the respective analysis.

The method of the present invention allows for a fast, simple and reliable assessment of the ability of a test fragrance ingredient or a test fragrance composition to improve the relaxation state of a human subject. Fragrances may be tested in wide variety of settings, from a non-motion laboratory setting to in-context-testing. Furthermore, the method allows for detecting sub-conscious effects, thereby avoiding common issues of conscious methods (e.g. interrogation), which often provide only limited and often inaccurate information due to dishonest responses, prior survey biases, and inarticulacy, for instance.

In order to qualify as a relaxing fragrance ingredient or fragrance composition, a test fragrance ingredient/composition must fulfil at least three of the Conditions 1-6. Preferably, at least four out of the Conditions 1-6 are met, more preferably at least five, and most preferably all six.

More specifically, the applicant has identified certain specific Channels, haemoglobin types and time points that are particularly indicative of the effect on the relaxation state of the human subject.

Therefore, in an embodiment, at least 10, more preferably at least 12, and most preferably at least 15, of the following conditions are met:
C1. Channel 6 shows a statistically significant increase of Deoxy Hb after 30 seconds of smelling;
C2. Channel 15 shows a statistically significant increase of Deoxy Hb after 30 seconds of smelling;
C3. Channel 3 shows a statistically significant increase of Deoxy Hb after 0-5 seconds of smelling;
C4. Channel 5 shows a statistically significant increase of Deoxy Hb after 0-5 seconds of smelling;
C5. Channel 8 shows a statistically significant decrease of Deoxy Hb after 0-5 seconds of smelling;
C6. Channel 15 shows a statistically significant increase of Deoxy Hb after 0-5 seconds of smelling;
C7. Channel 8 shows a statistically significant decrease of Deoxy Hb after 0-10 seconds of smelling;
C8. Channel 15 shows a statistically significant increase of Deoxy Hb after 0-10 seconds of smelling;
C9. Channel 5 shows a statistically significant increase of Deoxy Hb after 5-10 seconds of smelling;
C10. Channel 8 shows a statistically significant decrease of Deoxy Hb after 5-10 seconds of smelling;
C11. Channel 15 shows a statistically significant increase of Deoxy Hb after 5-10 seconds of smelling;
C12. Channel 20 shows a statistically significant decrease of Total Hb after 30 seconds of smelling;
C13. Channel 10 shows a statistically significant decrease of Total Hb after 0-5 seconds of smelling;
C14. Channel 12 shows a statistically significant decrease of Total Hb after 0-10 seconds of smelling;
C15. Channel 4 shows a statistically significant increase of Oxy Hb after 30 seconds of smelling;
C16. Channel 17 shows a statistically significant decrease of Oxy Hb after 30 seconds of smelling;
C17. Channel 15 shows a statistically significant decrease of Oxy Hb after 0-5 seconds of smelling;
C18. Channel 3 shows a statistically significant decrease of Oxy Hb after 5-10 seconds of smelling;
C19. Channel 16 shows a statistically significant decrease of Oxy Hb after 5-10 seconds of smelling;
C20. Channel 20 shows a statistically significant decrease of Oxy Hb after 5-10 seconds of smelling.

Most reliable results were observed for conditions C2, C4, C5, C6, C9, C11, C13, C15, C16, and C20.

Therefore, in an embodiment, at least 5, preferably at least 6, more preferably at least 7, even more preferably at least 8, still more preferably at least 9, and most preferably all 10, of the following conditions are met: C2, C4, C5, C6, C9, C11, C13, C15, C16, and C20.

Based on the above described method of assessment, it was possible to develop guidelines for creating fragrance compositions that have a relaxing effect on human subjects.

Therefore, the present invention also provides a method of creating a fragrance composition having a relaxing effect on a human subject, comprising the steps of:
(i) creating a test fragrance composition;
(ii) assessing the ability of the test fragrance composition to improve the relaxation state of a human subject according to the method described above; and
(iii) adjusting, if necessary, the test fragrance composition by adding and/or removing at least one fragrance ingredient and/or increasing and/or reducing the concentration of at least one fragrance ingredient until the fragrance composition is found to improve the relaxation state of the human subject.

Therefore, it is first assessed whether or not the test fragrance composition provides a relaxing effect. Subsequently, if necessary, the composition is adjusted to create an improved fragrance composition.

Steps (ii) and (iii) may be repeated if necessary and/or desired.

Increasing the level of relaxing ingredients (R) increases the likelihood that the fragrance composition would have a suitable character to deliver the relaxing benefit. Other ingredients reduce the likelihood that the benefit will be achieved, as their level in the fragrance composition is increased, i.e. they are non-relaxing (NR).

Therefore, in one embodiment, at least one relaxing fragrance material R is added to the (test) fragrance composition in step (iii).

Alternatively or in addition, at least one non-relaxing fragrance material NR may be removed from the (test) fragrance composition in step (iii).

Alternatively or in addition, the concentration of at least one relaxing fragrance material R may be increased in step (iii).

Alternatively or in addition, the concentration of at least one non-relaxing fragrance material NR may be reduced in step (iii).

It has been found that the following fragrance materials have a relaxing effect: musk ingredients, floral-orris ingredients, sweet-vanilla ingredients, sweet-heliotrope ingredients, sweet-tonka ingredients, woody-vetiver ingredients, 2-(phenoxy)ethyl 2-methylpropanoate (phenoxyethyl isobutyrate), 1-(2-naphtalenyl)-ethanone (oranger crystals), 2-(4-methylcyclohex-3-en-1-yl)propan-2-ol (terpineol), (E)-1-methoxy-4-(prop-1-en-1-yl)benzene (anethole), 2-ethyl-4(2′,2′,3′-trimethylcyclopent-3-enyl)but-enol (Bangalol or Radjanol), basil oil, (E)-3,7-dimethylnona-1,6-dien-3-ol (ethyl linalool), 2-(2′-methylpropyl)-4-hydroxy-4-methyltetrahydropyran (Florosa), (E)-3,7-dimethylocta-2,6-dien-1-ol (geraniol), methyl 2-aminobenzoate (methyl anthranilate), mixtures of 2-methyl-1-phenylpropan-2-yl butanoate and (phenoxy)ethyl 2-methylpropanoate (Prunella), 5-heptyldihydrofuran-2(3H)-one (peach pure), and mixtures thereof.

It has been found that the following fragrance materials have a non-relaxing effect: 2-methylundecanal (methyl nonyl aldehyde), prop-2-enyl 2-(3-methylbutoxy)acetate (allyl amyl glycolate), (Z)-1-((3aS,6R,7R,8aS)-6,8,8-trimethyloctahydro-3H-3a,7-methanoazulen-3-ylidene) propan-2-one (acetyl cedrene), 3a,6,6,9a-perhydrotetramethylnaphtho [2,1-b] furan (Amberlyn Super), pentyl 2-hydroxybenzoate (amyl salicylate), armoise oil, benzyl 2-hydroxybenzoate (benzyl salicylate), bergamot oil, 4-tert-butyl-3-phenylpropanal (Bourgeonal), cedar leaf oil, 3,7-dimethyloct-6-en-1-ol (citronellol), (E)-1-(2,6,6-trimethyl-1-cyclohexenyl)but-2-en-1-one (beta damascone, 2-methyl-1-phenylpropan-2-yl acetate (dimethyl benzyl carbinyl acetate), ethyl 2,6,6-trimethylcyclohexadienecarboxylate (Ethyl Safranate), methyl 2,4-dihydroxy-3,6-dimethylbenzoate (Evernyl or Everniate), (E)-3,7-dimethylocta-2,6-dienenitrile (geranyl nitrile), 3-(1,3-benzodioxol-5-yl)-2-methylpropanal (Helional or Tropional), (Z)-hex-3-enol, hexyl 2-hydroxybenzoate (hexyl salicylate), lemon oil, 2,4-dimethyl-3-cyclohexene-1-carbaldehyde (Cyclal C or Tricyclal or Ligustral), 3-(4-(1,1-dimethylethyl)phenyl-2-methylpropanal (Lilial), 4-(4-hydroxy-4-methylpentyl)cyclohex-3-enecarbaldehyde (Lyral), 3-methyl-5-phenylpentanol (Mefrosol), orange oil, orange terpenes, tagetes oil, 3,7-dimethyloctan-1-ol (pelargol), and mixtures thereof.

Where trivial names are used to describe useful perfume ingredients herein, the skilled perfumer will understand that these are commonly used names in the art of perfumery. However, the skilled perfumer would also understand that these ingredients may also be known by other trivial synonyms, by CAS registry numbers, or by more formal nomenclature, such as IUPAC nomenclature. Furthermore, the skilled perfumer would be familiar with these other trivial synonyms, as well as with more formal nomenclature, or at the least, would be aware of standard reference works, such as The Good Scents Company website, which contains a comprehensive list of trivial names, registry numbers and more formal nomenclature for the perfume ingredients contained in the perfumers' palette.

Perfume compositions and individual perfume ingredients may be characterized by their odour attributes. Although perfume creation is part science and part artistry, and there is no absolute prescribed definition for odour attributes of perfume compositions and perfume ingredients, nevertheless trained perfumers, realizing that there will be margin for some subjectivity, will be able to assign perfume compositions and ingredients to a general odour descriptor and an odour family.

Odour families provide a general description of an odour space, and their number is usually limited. Hence, most of the ingredients used in perfumery and particularly useful in the context of the present invention may be described by a small set of odour families selected from the group consisting of “aldehydic”, “ambery”, “animalic”, “aromatic/herbal”, “citrus”, “floral”, “fruity”, “green”, “musky”, “spicy”, “sweet”, “watery”, and “woody”.

Odour descriptors provide a more accurate description of the odour of a perfume composition or ingredient within a family. They are more abundant and their number and diversity is often unlimited. Examples of odour descriptors include, but are not limited to, “aldehyde zest”, “almond”, “amber dry”, “ambergris”, “anis tarragon”, “apple”, “armoise”, “balsam”, “banana”, “blackcurrant”, “butter”, “candied fruit”, “caraway seed”, “cedar”, “cinnamon”, “citronella”, “clove”, “cocoa”, “coconut”, “coniferous”, “cooked sugar”, “copaiba”, “coriander leaf”, “cucumber”, “eucalyptus”, “fecal”, “freesia”, “galbanum”, “grapefruit”, “grass”, “heliotrope”, “jasmine”, “lavender”, “leaf”, “leather”, “lemon”, “licorice-fenugreek”, “lily of the valley”, “lime”, “liquor”, “lychee”, “mandarin”, “mango”, “medicinal”, “melon”, “metallic”, “milk cream”, “molasses”, “moss”, “mushroom”, “musk”, “musk tonkin”, “nut”, “orange”, “orange flower”, “orris”, “passionfruit”, “patchouli”, “peach”, “pear”, “peppermint”, “pineapple”, “raspberry”, “rhubarb”, “rose”, “rosemary”, “sandalwood”, “sea water”, “solar”, “strawberry”, “terpenic”, “thyme”, “tonka”, “vanilla”, “vetiver”, “violet”, and “wax”.

This selection of odour families and odour descriptors allows the skilled perfumer to characterize the odour of all perfume ingredients contained in the perfumer's palette. Nevertheless, for the trained perfumer, reading the contents of this specification as a whole together with their common general knowledge, it would not present undue burden to modify part or all of this vocabulary around which there is subjectivity, and such modification would not impact the selection of perfume ingredients useful to positively impact the perception of well-being.

Specific examples of musk ingredients, floral-orris ingredients, sweet-vanilla ingredients, sweet-heliotrope ingredients, sweet-tonka ingredients, and woody-vetiver ingredients, respectively, will be provided below.

Throughout this application, the term “oil” is meant to encompass fully natural essential oils and extracts, as well as oils derived from natural essential oils and extracts, and modified essential oils and extracts that may comprise additional ingredients; irrespective of the extraction method.

The term “oil” is meant to further also encompass any reconstitution or mixture of ingredients that provides a similar odour impression to the corresponding essential oil.

As used throughout this application, the term “terpineol” refers to single isomers of terpineol (e.g. alpha terpineol), as well as to mixtures of two or more isomers of terpineol.

The present invention further provides fragrance compositions for improving the relaxation state of a human subject.

The fragrance composition comprises:
a) at least about 25% by weight in total of at least five relaxing fragrance materials R;
b) optionally up to about 45% by weight in total of non-relaxing fragrance materials NR, provided that the weight ratio of R to NR is at least 0.75;
c) optionally up to about 75% by weight in total of neutral fragrance materials N; and
d) optionally up to about 25% by weight in total of other fragrance materials M, provided that the weight ratio of R to (M+NR) is at least 0.75.

In the above formulation guidelines, all percentages are based on total weight of the fragrance materials constituting the fragrance composition. This means that solvents, diluents and other vehicles are not taken into account in the calculation.

The present invention is based on extensive testing of fragrance materials, by consumer testing and measurement of brain activity using EEG and fNIRS. Statistical analysis of the resulting data has allowed classifying the fragrance materials into different categories:
relaxing fragrance materials or ingredients (R) induce in subjects exposed to them positive, low activation moods and emotions, such as relaxation (i.e. they exhibit relaxing properties);
non-relaxing fragrance materials or ingredients (NR) induce in subjects exposed to them negative, high activation moods or emotions (i.e. the exhibit non-relaxing properties); and
neutral fragrance materials or ingredients (N) have a neutral effect in terms of relaxing properties.

Other fragrance materials (of which there are several thousand currently available commercially and used in perfume formulation) are designated as class M materials.

The relaxing fragrance materials R are as defined above.

The non-relaxing fragrance materials NR are as defined above.

The neutral fragrance materials N are selected from the group consisting of benzyl acetate, cassis base, 2-methyl-3-(4-(1-methylethyl)phenyl)propanal (cyclamen aldehyde), (5R)-2-methyl-5-prop-1-en-2-ylcyclohex-2-en-1-one (carvone), (E)-3-phenylprop-2-en-1-ol (cinnamic alcohol), 2-methoxy-4-propylphenol (dihydroeugenol), 2,6-dimethyloct-7-en-2-ol (dihydromyrcenol), 4-allyl-2-methoxyphenol (eugenol), galbanum, 5-hexyloxolan-2-one (gamma decalactone), 7-hydroxy-3,7-dimethyloctanal (hydroxycitronellal), 1H-indole, (E)-2-methoxy-4-(prop-1-en-1-yl)phenol (isoeugenol), jasmin oil, 3-pentyltetrahydro-2H-4-pyranyl ethanoate (Jasmopyrane Forte), 3,7-dimethylocta-1,6-dien-3-ol (linalool), 3,7-dimethylocta-1,6-dien-3-yl acetate (linalyl acetate), methyl 3-oxo-2-pentylcyclopentaneacetate (methyl dihydrojasmonate), patchouli oil, 2-phenylethyl alcohol, 4-methyl-2-(2-methylprop-1-en-1-yl)tetrahydro-2H-pyran (rose oxide), rose oil, 5-(2,2,3-trimethyl-3-cyclopentyl-3-methylpentan-2-ol (Sandalore), 3-[5,5,6-trimethylbicyclo[2.2.1]hept-2-yl]cyclohexan-1-ol (Sandela), 1-phenylethyl acetate (styrallyl acetate), ylang-ylang, and mixtures thereof.

Cassis base is a reconstitution of, i.e. a mixture of fragrance ingredients resembling the smell of, cassis.

The other fragrance materials M comprises fragrance materials not included in the above, excluding odourless or low-odour solvents or diluents used as vehicles for fragrance materials.

Therefore, materials of class M include prior art fragrance materials, which are not specified herein as being members of any of classes R, NR or N, excluding odourless or low-odour solvents or diluents, as noted above. They may be single ingredients or mixtures, both synthetic and natural (for example essential oils), and are well described e.g. in: “Common Fragrance and Flavor Materials” by Bauer, Garbe and Surburg, VCH Publ., 2^{nd} edition (1990), and “Perfume and Flavour Materials”, Steffen Arctander, published in two volumes by the author (1969), also by Arctander “Perfume and Flavor Materials of Natural Origin” (1960), and Perfume & Flavor Chemicals”, S. Arctander (Allured Publishing, 1994), as well as later editions of this work, which perfume ingredients contained therein are herein incorporated by reference.

Perfume compositions of the present invention may further contain substantially odourless ingredients. In the context of the present invention, “substantially odourless” means that the ingredient has no odour or that its odour is weak and often barely perceptible. These substantially odourless ingredients include excipients conventionally used in conjunction with perfume ingredients in perfume compositions, for example carrier materials, and other auxiliary agents commonly used in the art, e.g. solvents, such as dipropylene glycol (DPG), isopropyl myristate (IPM), benzyl benzoate (BB), propylene glycol (PG) and triethyl citrate (TEC); mineral oils and vegetable oils; and antioxidants. As such, these substantially odourless ingredients are not considered to be perfume ingredients in the context of the present invention. In particular, solvents are not taken into account when calculating the weight percentages.

The perfume compositions of the present invention may be presented in the form of free-oil, or they may be encapsulated. Several encapsulating media are known in the art for encapsulating perfume compositions. Particular encapsulating media include microcapsules formed of aminoplast resins, such as melamine-formaldehyde resins, polyurea, polyamide, as well as copolymers of acrylic acid, methacrylic acid and their esters. Alternatively, the encapsulating media may be formed of natural or modified natural polymers, such as polysaccharides or proteins.

The above definition of the fragrance compositions of the present invention provides sufficient freedom in formulation to permit consideration of the hedonic properties of the composition. The invention can thus enable formulation of fragrance compositions that are relaxing and also have good hedonic properties.

The present invention describes how to formulate reliably fragrance compositions which are likely to induce or be associated with positive, low activation moods and emotions, particularly relaxing effects. The effects are sufficiently pronounced that they can be measured reliably and reproducibly. The fragrance compositions made according to the teachings disclosed herein can be hedonically pleasant, suitable for a wide range of consumer products, and of sufficient pleasantness/acceptability that they would be appropriate even if they did not possess added functionality. In addition, fragrance compositions of the invention can be resilient to variation in the target consumer group (e.g. Japanese vs. American), and have been found to be perceived as consistently relaxing/reassuring etc. for consumers in England, France, USA and Japan, for example.

Fragrance compositions in accordance with the invention have been found:
a) to promote a reduction in the amplitude of alpha wave activity, as measured by EEG;
b) to promote positive mood states such as relaxation. Test subjects have reported that they feel more relaxed after smelling or using consumer products incorporating the fragrance compositions, and that the products themselves smell more relaxing;
c) to promote calming, relaxed, mindful, balanced, caring, reassuring, safe mood states;
d) not to promote negative mood states, such as depressing, stressful, annoying, or bored mood states.

Increasing the level of relaxing ingredients (R) increases the likelihood that the fragrance would have a suitable character to deliver the relaxing benefit. Other ingredients reduce the likelihood that the benefit will be achieved as their level in the fragrance composition is increased, i.e. they are non-relaxing (NR). A third group (N) has a neutral effect. Combinations of fragrance ingredients, which are commercially useful and hedonically pleasant, generally require ingredients in all three classes to be present. Other perfume ingredients not assigned to any of the three groups (M) may be added to the fragrance compositions of the invention without loss of benefit, provided that the weight ratio of relaxing ingredients to the sum of M plus NR ingredients is equal to or greater to about 0.75.

In an embodiment, the fragrance composition comprises at least about 35%, more preferably at least about 45%, by weight in total of relaxing fragrance materials R.

In an embodiment, the fragrance composition comprises at least eight relaxing fragrance materials R, more preferably at least ten relaxing fragrance materials R. By increasing the number of fragrance ingredients, the hedonics of the fragrance composition are improved.

In an embodiment, the fragrance composition comprises up to 35%, preferably up to 25%, more preferably up to 20%, and most preferably up to 15%, by weight in total of non-relaxing fragrance materials NR.

In an embodiment, the fragrance composition comprises one or more musk ingredients selected from the group consisting of 1,4-dioxacycloheptadecane-5,17-dione (ethylene brassylate), 4,6,6,7,8,8-hexamethyl-1,3,4,6,7,8-hexahydrocyclopenta[g]isochromene (Galaxolide), mixtures of cyclohexadecanolide and cyclopentadecanone (Silvanone), 1-(1,1,2,6-tetramethyl-3-propan-2-yl-2,3-dihydroinden-5-yl)ethanone (Traseolide), cyclohexadecanolide, cyclopentadecanone, 17-oxacycloheptadec-6-en-1-one (ambrettolide), (9Z)-cycloheptadec-9-en-1-one (Civettone), (E)-3-methyl-5-cyclotetradecen-1-one (Cosmone), 6-acetyl-1,1,3,4,4,6-hexamethyltetrahydro-naphtha-lene (Extralide), 1-(1,1,2,3,3,6-hexamethyl-2H-inden-5-yl)ethanone (Fixolide), (12E)-1-oxacyclo-hexadec-12-en-2-one (Habanolide), [2-[1-(3,3-dimethylcyclohexyl)ethoxy]-2-methylpropyl]propanoate (Helvetolide), octahydrohexamethyl naphthoxirene (Moxalone), (5E)-3-methyl-cyclopentadec-5-en-1-one (Muscenone), 3-methyl-1-cyclopentadecanone (Muscone), 4-tert-butyl-2,6-dimethyl-3,5-dinitroacetophenone (musk ketone), 1,7-dioxacycloheptadecan-8-one (musk R1), (10Z)-13-methyl-1-oxacyclopentadec-10-en-2-one (Nirvanolide), 2-[1-(3,3-dimethylcyclohexyl)ethoxy]-2-methylpropyl cyclopropanecarboxylate (Serenolide), (3′E)-2-((3′,5′-dimethylhex-3′-en-2′-yl)oxy)-2-methylpropyl cyclopropanecarboxylate (Sylkolide), 1,15-pentadecanolide (Thibetolide), (5Z)-cyclohexadec-5-en-1-one (Velvione), and mixtures thereof.

In an embodiment, the fragrance composition comprises one or more floral-orris ingredients selected from the group consisting of N-1-(2,6,6-trimethyl-1-cyclohex-2-enyl)pent-1-en-3-one (Isoraldeine), 4-(2,6,6-trimethyl-1-cyclohex-2-enyl)pentanal (Cetonal), (E)-1-(2,6,6-trimethylcyclo-hex-2-en-1-yl)hepta-1,6-dien-3-one (cetone V), 4-(2,6,6-trimethylcyclohex-1-en-1-yl)butan-2-one (dihydro-ionone beta), 7,9-dimethylspiro[5.5]undecan-3-one (Dispirone), (E)-4-(2,6,6-trimethyl-cyclohex-1-en-1-yl)but-3-en-2-one (ionone beta), (E)-4-(2,6,6-trimethylcyclohex-2-en-1-yl)but-3-en-2-one (irisone alpha), (E)-4-(2,5,6,6-tetramethylcyclohex-2-en-1-yl)but-3-en-2-one (irone alpha), (E)-3-methyl-4-(2,6,6-trimethylcyclohex-2-en-1-yl)but-3-en-2-one (Isoraldeine cetone alpha or alpha-iso-methyl ionone), (Z)-3,4,5,6,6-pentamethylhept-3-en-2-one (Koavone), and mixtures thereof.

In an embodiment, the fragrance composition comprises one or more sweet-vanilla ingredients selected from the group consisting of benzoin resinoids, 3-ethoxy-4-hydroxybenzaldehyde (ethyl vanillin), 4-formyl-2-methoxyphenyl 2-methylpropanoate (Isobutavan), 4-hydroxy-3-methoxybenzaldehyde (vanillin), 2-methoxy-4-methylphenol (creosol), 2-ethoxy-4-(methoxymethyl)phenol (methyl diantilis), 2-propoxy-4-(methoxymethyl)phenol (propyl diantilis), 2-ethoxy-4-methylphenol (Ultravanil), and mixtures thereof.

In an embodiment, the fragrance composition comprises one or more sweet-heliotrope ingredients selected from the group consisting of 1-(4-methoxyphenyl)ethanone (acetanisole), 1-phenylethanone (acetophenone), 4-methoxybenzaldehyde (aubepine para cresol), benzo[d][1,3]dioxole-5-carbaldehyde (heliotropine), 1-(p-tolyl)ethanone (methyl acetophenone), and mixtures thereof. In an embodiment, the fragrance composition comprises one or more sweet-tonka ingredients selected from the group consisting of 2H-chromen-2-one (coumarin), octahydro-2H-chromen-2-one (bicyclononalactone), dec-9-en-1-yl (E)-3-(2-hydroxyphenyl)acrylate (Scentaurus Tonkarose), tonka bean oil, resinoid, extract or balsam, tonka roasted absolutes, coumarin replacers (e.g. Coumarex DB), and mixtures thereof.

In an embodiment, the fragrance composition comprises one or more woody-vetiver ingredients selected from the group consisting of vetiver oil, (4,8-dimethyl-2-propan-2-ylidene-3,3a,4,5,6,8a-hexahydro-1H-azulen-6-yl) acetate (Vetiveryl Acetate), and mixtures thereof.

The above groups of relaxing fragrance ingredients may be combined as desired.

In an embodiment, the weight ratio of R to NR is at least 1:1, more preferably at least 2:1, and most preferably at least 3:1.

In an embodiment, the weight ratio of R to (M+NR) is at least 1:1, more preferably at least 2:1, and most preferably at least 3:1.

Another aspect of the invention relates to a method of delivering positive mood benefits, particularly relaxation, to human subjects, comprising delivering the fragrance composition to said human subjects. For instance, the fragrance may be delivered in a consumer product.

Therefore, the present invention also provides a consumer product comprising the fragrance composition of the invention.

The perfume compositions of the present invention may be used to impart desirable odour impressions on all manner of consumer products, such as for instance hydro-alcoholic perfumes, deodorants, antiperspirants, skin care products, hair care products, laundry care products, home care products or air fresheners.

More particularly, the perfume compositions of the present invention may be employed in laundry care applications, personal care products for treating the hair and/or skin of human subjects, oral care products, and air care products.

Consumer products comprise formulated mixtures of various functional ingredients, such as surfactants, emulsifiers, polymers, fillers and solvents. These formulated mixtures are usually referred to as “bases”.

Particular consumer products include, but are not limited to consumer products intended for application to the body (i.e. skin or hair), to hard surfaces (e.g. kitchen and bathroom worktops, ceramic surfaces), to fabrics, and for air care benefits (e.g. air-fresheners). Such products can take a variety of forms, including, but not limited, to powders, bars, sticks, tablets, creams, mousses, gels, liquids, sprays and sheets. The proportion of perfume composition contained in such products may lie in a range from 0.05% (as for example in a low odour skin cream) to 100 wt.-% (as for example in an air freshener) based on the total weight of the consumer product. The means of incorporating a perfume composition into a consumer product is known. Existing techniques may be used for incorporating the perfume composition directly into a product, or the perfume composition may be absorbed on a carrier material and then admixed to the product.

In an embodiment of the present invention, the consumer product is a laundry care product. Laundry care products include powder and liquid detergents and fabric softeners, stain removers and pre-wash treatments, conditioners and softeners (including standard and concentrated conditioners, softeners and dryer sheets), laundry aids (including stain removers, ironing aids, whiteners and colour care products and other ancillary fabric care products), laundry detergents (including machine wash liquid detergents, other machine wash detergents—including powders, capsules and tablets—and hand wash detergents—powders, flakes and cakes/bars), sheet sprays, clothing sprays, laundry perfumes, dryer sachets, perfumed sachets, dryer sheets, laundry soap, laundry detergents, detergent for delicate textiles, ironing sprays, starch, perfume sheets, pillow mists, drawer liner sheets, cedar closet sprays, linen waters, and refills and combinations thereof.

In an embodiment of the invention, the consumer product is a personal care product. Personal care products include soaps, shower gels, body creams, body lotions, body mists, perfumery, cosmetics, floating bath oils, after shaves, creams, lotions, deodorants (including stick deodorants), pre-electric shave lotions, after-shave lotions, antiperspirants, shampoos, conditioners, rinses, skin care products, eye makeups, body shampoos, protective skin formulations, lipsticks, lip glosses, after-bath splashes, pre-sun and sun products (including sunscreens). Virtually any chemical product which comes into contact with the hair or skin and which may include effective amounts, concentrations or proportions of one or more of the perfume compositions of the present invention may be considered a personal care product according to the present invention.

In an embodiment of the present invention, the consumer product is an air care product. Air care products include candles and air-freshener devices, such as liquid electrical air-freshener devices, aerosol sprays, pump action sprays, perfumed candles, membrane permeation devices, liquid wick devices, oil based gel perfumes, and aqueous gels.

In an embodiment of the present invention, the consumer product is a home care product. Home care products can be used particularly for cleaning, rinsing, care or treatment of industrial, domestic or communal hard surfaces, as well as textile article surfaces; they are targeted at conferring on the surfaces treated therewith benefits such as water repellence, soil release, stain resistance, anti-fogging, surface repair, anti-wrinkling, shine, lubrication and/or at improving the residuality, impact and/or efficacy of active materials comprised in said home care product. Hence, home care compositions according to the invention include surface cleaning compositions (for example glass, floor, counter, bath, toilet bowl, sink, appliance and furniture cleaning compositions), disinfectants (for example spray and solid air disinfectants, including gels, and spray, solid, liquid and paste surface disinfectants), waxes and other surface protecting and/or polishing compositions, and rug shampoos.

Also included within the scope of the invention is a method of delivering positive mood benefits or relaxation benefits to a subject, particularly a human, comprising administering to the subject an effective amount of a fragrance composition in accordance with the invention. The composition should be administered in an appropriate amount to produce a benefit (i.e. a suprathreshold amount) without causing irritation (i.e. a non-irritant amount). An appropriate effective amount of any given composition can be readily determined, e.g. by experiment. To be effective, the compositions should be administered for inhalation by the subject.

Therefore, the present invention also provides a method of improving the relaxation state of a human subject, comprising the step of providing an effective amount of the fragrance composition of the invention to the human subject.

In the context of the studies resulting in the present invention, several fragrance ingredients have been identified that are able to improve the relaxation state of a human subject.

Therefore, the present invention also relates to the use of a fragrance ingredient for improving the relaxation state of a human subject, wherein the fragrance ingredient is selected from the group consisting of 17-oxacycloheptadec-6-en-1-one (ambrettolide), (9Z)-cycloheptadec-9-en-1-one (Civettone), (E)-3-methyl-5-cyclotetradecen-1-one (Cosmone), 1-(1,1,2,3,3,6-hexamethyl-2H-inden-5-yl)ethanone (Fixolide), (12E)-1-oxacyclohexadec-12-en-2-one (Habanolide), [2-[1-(3,3-dimethylcyclohexyl)ethoxy]-2-methylpropyl]propanoate (Helvetolide), octahydrohexamethyl naphthoxirene (Moxalone), (5E)-3-methylcyclopentadec-5-en-1-one (Muscenone), 3-methyl-1-cyclopentadecanone (Muscone), 4-tert-butyl-2,6-dimethyl-3,5-dinitroacetophenone (musk ketone), 1,7-dioxacycloheptadecan-8-one (musk R1), (10Z)-13-methyl-1-oxacyclopentadec-10-en-2-one (Nirvanolide), 2-[1-(3,3-dimethylcyclohexyl)ethoxy]-2-methylpropyl cyclopropanecarboxylate (Serenolide), (3′E)-2-((3′,5′-dimethylhex-3′-en-2′-yl)oxy)-2-methylpropyl cyclopropanecarboxylate (Sylkolide), 1,15-pentadecanolide (Thibetolide), (5Z)-cyclohexadec-5-en-1-one (Velvione), 1-(2,6,6-trimethyl-1-cyclohex-2-enyl)pent-1-en-3-one (Isoraldeine), 4-(2,6,6-trimethyl-1-cyclohex-2-enyl)pentanal (Cetonal), (E)-1-(2,6,6-trimethylcyclohex-2-en-1-yl)hepta-1,6-dien-3-one (cetone V), 4-(2,6,6-trimethylcyclohex-1-en-1-yl)butan-2-one (dihydro-ionone beta), 7,9-dimethylspiro[5.5]undecan-3-one (Dispirone), (E)-4-(2,6,6-trimethylcyclohex-1-en-1-yl)but-3-en-2-one (ionone beta), (E)-4-(2,6,6-trimethylcyclohex-2-en-1-yl)but-3-en-2-one (irisone alpha), (E)-4-(2,5,6,6-tetramethylcyclohex-2-en-1-yl)but-3-en-2-one (irone alpha), (E)-3-methyl-4-(2,6,6-trimethylcyclohex-2-en-1-yl)but-3-en-2-one (Isoraldeine cetone alpha), (Z)-3,4,5,6,6-pentamethylhept-3-en-2-one (Koavone), benzoin resinoids, 3-ethoxy-4-hydroxybenzaldehyde (ethyl vanillin), 4-formyl-2-methoxyphenyl 2-methylpropanoate (Isobutavan), 4-hydroxy-3-methoxybenzaldehyde (vanillin), 2-methoxy-4-methylphenol (creosol), 2-ethoxy-4-(methoxymethyl)phenol (methyl diantilis), 2-propoxy-4-(methoxymethyl)phenol (propyl diantilis), 1-(4-methoxyphenyl)ethanone (acetanisole), 1-phenylethanone (acetophenone), 4-methoxybenzaldehyde (aubepine para cresol), benzo[d][1,3]dioxole-5-carbaldehyde (heliotropine), 1-(p-tolyl)ethanone (methyl acetophenone), octahydro-2H-chromen-2-one (bicyclononalactone), dec-9-en-1-yl (E)-3-(2-hydroxyphenyl)acrylate (Scentaurus Tonkarose), tonka bean oil, resinoid, extract or balsam, tonka roasted absolutes, coumarin replacers (e.g. Coumarex DB), and mixtures thereof.

The present invention is further illustrated by means of the following non-limiting examples:

### EXAMPLE 1: FUNCTIONAL NEAR INFRARED SPECTROSCOPY TESTING

### Task

The experimental protocol was divided in two parts:

In the first one, participants smelled a series of at least twelve (three consecutive repetitions of four different odour conditions) or fifteen (three consecutive repetitions of five different odour conditions) fragrance samples provided on sorbarods, while their brain activity was being monitored through a fNIRS cap placed on the forehead. fNIRS channels were arranged as shown in FIG. 1.

In the second part, they rated each fragrance for the dimensions of pleasantness, invigorating power, relaxing power, familiarity and strength of the odour using a questionnaire. During this second part, their brain activity was not monitored.

The samples were typically prepared as follows: In each sorbarod, 0.8 g of neat fragrance oil was placed in the cotton insert by means of a pipette. Previous tests demonstrated that a range between 0.75 g and 0.85 g of neat oil will not significantly alter the perception of the fragrance in terms of properties and intensity, therefore the range is acceptable for any brain imaging test without influencing the results. Once the oil was dropped, the plastic cap was immediately placed on the sorbarod to prevent any diffusion of the fragrance in the environment. Sorbarods were then kept in standing (vertical) position for at least 24 h before being used for the test. After the sorbarod rests for 24 h, the full insert becomes soaked with the oil, guaranteeing that, if adequately stored (i.e. without exposing the sorbarod to direct sunlight or to extremely high temperatures, above 35° C.), the fragrance oil maintains the same olfactive properties (characteristics and intensity) for at least four weeks, up to eight weeks, depending on the oil. In this time frame, the sorbarods can be used for brain imaging tests without any significant alteration of the results. In the tests described in the current document, the samples were used within two weeks from the day they were created. Samples were normally stored in a refrigerator at 4° C. from the moment they were made to the morning of the test. The experimenter made sure that sorbarods were taken out of the fridge at least 2 h before the test to ensure they reached room temperature before being used. Removing them from the fridge the evening before the test, and leaving them overnight at room temperature, also does not have any significant effect on test results, as previous trials demonstrated.

In the first part, participants were asked to smell, keeping their eyes closed, the proposed number of sorbarods. They were not required to complete any other tasks, in order to eliminate any possible source of confound in the data not related to the perception of odours. In each test, one of the samples contained the fragranced benchmark and another one did not contain any fragrance (control sample). The other two or three samples contained the test fragrances. Thanks to the three repetitions, it was possible to confirm that the overall results were not affected by the number of fragrances tested in one trial, i.e. that the test of four or five different conditions in a single test was completely equivalent and that the studies were fully comparable.

The order of presentation for the sorbarods was semi-randomized: the order of the fragrances in each block was fully randomized, however participants had to complete smelling all samples in a block before moving to the following one, and the first sample of each block was always different from the last of the previous one, so to avoid smelling the same fragrance twice in two consecutive assessments.

After the participants had smelled all the sorbarods, the fNIRS cap was removed from the head and they completed the questionnaires at a self-timed pace, meaning that they were able to smell again each sample as many times as they wanted and take all the time they needed to answer each question. For this reason, no specific timeline for the second part of the experimental procedure will be reported in the following section.

### Timeline

All participants completed three blocks of four or five samples each. The three blocks were consecutive, and the participants were unaware that the sequence of four or five samples was repeated three times, as they were only told that the test involved smelling twelve or fifteen samples. Participants were asked, for each sample, to take the sorbarod in their hand, close their eyes, smell the sorbarod for thirty seconds and then, after returning the sorbarod to the experimenter, rest for thirty seconds with the eyes open. Longer intervals between two consecutive samples were taken if the participant explicitly asked for it, or if the fNIRS signals were not at a baseline level (necessary condition to start with a new trial). The latter case would happen in case of heavy movements from the participants, such as sneezing; however, the recovery time was in the order of a few seconds.

### Participants

For each study, at least fifteen healthy adults took part in this experiment. No specific selection criteria (i.e. handedness, age, etc.) have been applied in the choice of the participants, since no relevant exclusion criteria have been identified prior to testing.

### Statistical Analysis

Statistical significance was verified using a 2-tailed Student's t-test with a statistical significance threshold at 0.05.

### EXAMPLE 2: MOOD PORTRAITS® TESTING

### Task

Mood Portraits® is a self-report nonverbal method using pictures to measure consumers' moods and emotional responses to fragrances. This method allows participants to express what they feel in response to smelling a fragrance by selecting images that match their feelings rather than verbalising and rating their thoughts and emotions.

The experimental protocol was divided in two parts. In the first one, participants smelled a series of fifteen sorbarods and, while smelling each one, they selected a number of pictures chosen from a set of thirty pictures to describe the fragrance. The thirty pictures, printed in color on A4 laminated sheets, were arranged on a display board. The number of pictures chosen by each participant to describe the fragrances was not pre-determined: each participant could choose as many as they wanted to describe each fragrance. The minimum number of pictures they had to select was one. In the second part of the test, they rated each fragrance for the dimensions of pleasantness, invigorating power, relaxing power, familiarity and strength of the odour using a questionnaire.

The order of presentation for the sorbarods was fully randomised and the pictures were arranged on four different boards to create a randomisation of the layout.

### Timeline

All participants smelled and rated fifteen fragrances during a single session. There was no time limit for the participants to smell the fragrance nor to select the pictures associated to each fragrance. This allowed the participants to provide truer responses without any time pressure associated.

Participants were allowed breaks at their leisure to prevent any fatigue or carry over effect, and moved to the following fragrance only when they considered themselves ready.

### Participants

For each test involving fifteen fragrances, a hundred healthy adults were asked to participate in the study. Participants were screened for olfactive impairment, respiratory conditions or other personal conditions that could alter their sense of smell (e.g. pregnancy or consumption of tobacco-based products, like cigarettes). No other selection criteria (i.e. handedness, age, gender, etc.) have been applied in the choice of the participants, since no relevant exclusion criteria have been identified prior to testing.

### EXAMPLE 3: COMPOSITIONS TESTED

| | | A | B | C | D | E | F | G |
|---|---|---|---|---|---|---|---|---|
| Ingredient | Group | wt % | wt % | wt % | wt % | wt % | wt % | wt % |
| BENZYL BENZOATE | INERT | 1.0000 | | | | | | |
| DIPROPYLENE GLYCOL | INERT | 0.5400 | 32.706 | 8.6000 | 1.8000 | 16.1444 | 33.9000 | |
| DOWANOL TPM | INERT | | | 0.1000 | | | | 0.1000 |
| ISOPROPYL MYRISTATE | INERT | 8.7000 | 0.495 | 0.09 | | | | 0.09 |
| TRIETHYL CITRATE | INERT | | 0.9 | 0.1683 | | | | 0.1683 |
| BENZYL ACETATE | N | 0.5000 | 0.8 | | | | 1.5000 | |
| CASSIS BASE | N | | | 1.4000 | | 0.3333 | 0.5000 | 1.4000 |
| CINNAMIC ALCOHOL | N | 0.8000 | | | | | | |
| CYCLAMEN ALDEHYDE | N | | 1.2 | | | | 1.5000 | |
| EUGENOL | N | 0.1000 | | | | | | |
| GARDENOL | N | 0.5000 | | | | | 3.0000 | |
| HEDIONE | N | 6.7000 | 11.2 | 4.5000 | 8.0000 | 27.7911 | 15.0000 | 4.5000 |
| HYDROXYCITRONELLAL | N | | | | | | 5.0000 | |
| INDOLE | N | | | | | | 0.0500 | |
| ISOEUGENOL | N | | | | | | 1.0000 | |
| JASMIN OIL | N | | | | | 0.0222 | 1.0000 | |
| JASMOPYRANE FORTE | N | | 1.1 | | | | | |
| LINALYL ACETATE | N | | | | | 6.6667 | | |
| PATCHOULI OIL | N | | | 2.7000 | | 0.0333 | | 2.7000 |
| PHENYL ETHYL ALCOHOL | N | 0.5200 | | 5.0500 | | 0.3333 | 2.0000 | 5.0500 |
| ROSE OIL | N | 0.0100 | | | | | | |
| ROSE OXIDE CO | N | | | 0.5000 | | | | 0.5000 |
| SANDALORE | N | 1.4000 | | | | | | |
| SANDELA | N | | 0.255 | | | | | |
| YLANG YLANG | N | | | | | | 1.0000 | |
| ALDEHYDE C 12 MNA | NR | | | | | 0.0111 | | |
| AMBROFIX | NR | | 0.015 | 2.0000 | | | | 2.0000 |
| BENZYL SALICYLATE | NR | 1.6000 | | 7.9550 | 6.0000 | 4.4444 | | 7.9550 |
| BERGAMOT OIL | NR | 1.0000 | | | 12.0000 | | | |
| BOURGEONAL | NR | | 0.8 | | | | | |
| CITRONELLOL | NR | 0.5300 | | 4.0000 | | 0.1111 | 1.0000 | 4.0000 |
| CYCLOHEXAL | NR | 4.8000 | | | 8.0000 | | | |
| DAMASCONE BETA | NR | 0.0300 | | | | | | |
| DIMETHYL BENZYL CARBINYL ACETATE | NR | 0.5000 | | | | | | |
| ETHYL SAFRANATE | NR | | | 2.4000 | 0.1000 | | | 2.4000 |
| EVERNYL | NR | | | 0.7000 | | | | 0.7000 |
| HEXENOL-3-CIS | NR | | | 0.7000 | 0.0600 | 0.1111 | | 0.7000 |
| HEXYL SALICYLATE | NR | | 5.4 | | | | | |
| LEMON OIL | NR | | | | 3.0000 | | | |
| LILIAL | NR | 4.8000 | | 1.0000 | | | | 1.0000 |
| MEFROSOL | NR | | 5.8 | | | | | |
| ORANGE OIL | NR | | | 3.0000 | | | | 3.0000 |
| TRICYCLAL | NR | | | 1.5000 | 0.0400 | 0.2222 | | 1.5000 |
| TROPIONAL | NR | | | | | 4.4444 | | |
| AUBEPINE PARA CRESOL | R | | 0.9 | | | | | |
| BICYCLO NONALACTONE | R | | 0.5 | | | | | |
| CETONE V | R | | | | | | 0.0500 | |
| COUMARIN | R | | | | | | 0.2500 | |
| ETHYL LINALOOL | R | 0.0200 | 3 | | | 5.5556 | 10.0000 | |
| ETHYL VANILLIN | R | 0.5000 | 0.2 | 0.0500 | | | 1.0000 | 0.0500 |
| ETHYLENE BRASSYLATE | R | | 6.9 | | 18.0000 | 5.5556 | 5.0000 | |
| FIXOLIDE | R | 6.7000 | | | | | | |
| FLOROSA | R | | 6 | | | 1.6667 | | |
| GALAXOLIDE | R | 17.0100 | | | | | | |
| GERANIOL | R | 0.0600 | | 2.0000 | | | | 2.0000 |
| HABANOLIDE | R | | 2.5 | | | | | |
| HELIOTROPINE | R | 1.9000 | 0.8 | | 3.0000 | | 0.5000 | |
| IONONE BETA | R | | | 2.0000 | | | | 2.0000 |
| IRISONE ALPHA PRIME GRL | R | 1.0000 | | | | | | |
| ISORALDEINE | R | 19.5200 | | | 12.0000 | 0.5556 | | |
| METHYL ANTHRANILATE | R | | | | | | 2.0000 | |
| MUSK KETONE | R | 0.5000 | | | | | | |
| MUSK R1 | R | | 0.2 | | | | | |
| PEACH PURE | R | 0.3000 | 0.04 | | | | 0.2000 | |
| PRUNELLA | R | 2.0000 | | | 8.0000 | | | |
| RADJANOL | R | | 0.4 | | 6.0000 | 0.5556 | | |
| SCENTAURUS TONKAROSE | R | | | 0.0600 | | | | 0.0600 |
| SILVANONE SUPRA | R | | 3.2 | | 4.0000 | 7.7778 | | |
| SYLKOLIDE | R | | | | | | 0.5000 | |
| TERPINEOL | R | | 3.6 | | | | | |
| THIBETOLIDE | R | | 1.8 | 4.0000 | | | | 4.0000 |
| VANILLIN | R | 1.0000 | | | 3.0000 | | | |
| AGRUMEX | | | | | | 0.4444 | | |
| ALDEHYDE C 10 DECYLIC | | | | 1.0000 | | | | 1.0000 |
| ALDEHYDE C 11 UNDECYLENIC | | | | 1.2000 | | | | 1.2000 |
| ALDEHYDE C 110 UNDECYLIC | | | 0.08 | | | | | |
| ALDEHYDE C 12 LAURIC | | | 0.08 | | | | | |
| ALDEHYDE C 9 NONYLIC | | | 0.05 | | | | | |
| ALDEHYDE ISO C 11 | | | 0.002 | | | | | |
| AMBERMAX | | | | 0.1000 | | | | 0.1000 |
| BENZYL ACETONE | | | | 1.0000 | | | | 1.0000 |
| BIGARYL | | | | 0.0002 | | | | 0.0002 |
| BORNYL ACETATE LIQUID | | | | | | 0.1111 | | |
| CALONE LIQUID | | | | | | 0.0089 | | |
| CASHMERAN | | | | | | | 0.5000 | |
| CASSYRANE | | | | 0.0200 | | | | 0.0200 |
| CITRAL LEMAROME N | | | | | | 0.0778 | | |
| CITRONELLAL | | | | | | 0.0111 | | |
| CITRONELLYL ACETATE | | | 1.8 | | | | | |
| CORNMINT OIL DEMENTHOLIZED | | | | | | 0.5556 | | |
| CYCLOGALBANATE | | 0.1000 | | | | | | |
| CYCLOHEXYL SALICYLATE | | | 1.2 | | | | | |
| DAMASCENONE | | | 0.08 | | | | | |
| DAMASCONE DELTA | | 0.0300 | | 1.5000 | | | 0.0500 | 1.5000 |
| DECENAL-4-TRANS | | | | 0.0800 | | | | 0.0800 |
| DUPICAL | | | 0.1 | | | | | |
| EBANOL | | | 0.2 | | | | | |
| ETHYL ACETOACETATE | | | | | | 0.3333 | | |
| ETHYL LINALYL ACETATE | | | | | | 5.5556 | | |
| FENNALDEHYDE | | | 0.2 | | | | | |
| FLORALOZONE | | | | 1.2000 | | | | 1.2000 |
| FLORHYDRAL | | | | 1.1000 | | | | 1.1000 |
| GALBANONE 10 | | | | | | 0.1333 | | |
| GERANIUM OIL | | 0.0100 | | 1.0000 | | | 0.2000 | 1.0000 |
| HEXENYL-3-CIS ACETATE | | | 0.04 | | | 0.0444 | | |
| HEXENYL-3-CIS SALICYLATE | | | 1.5 | 2.0000 | 3.0000 | 1.6667 | 2.5000 | 2.0000 |
| HEXYL CINNAMIC ALDEHYDE | | | | 3.0000 | | 3.6667 | | 3.0000 |
| INDOFLOR | | | 0.001 | | | | | |
| ISO E SUPER | | 14.3200 | | | | | 7.5000 | |
| INDOLENE | | | | | | 0.0556 | | |
| ISOJASMONE | | | | | | | 0.3000 | |
| ISOMENTHONE DL | | | | 0.5000 | | | | 0.5000 |
| JASMACYCLENE | | | | 2.5000 | | | | 2.5000 |
| JASMIN OIL | | 0.3000 | | | | | | |
| JASMONE CIS | | | | | | 0.2222 | | |
| KARANAL | | | | 0.4000 | | | | 0.4000 |
| LABIENOXIME | | | | 0.0015 | | | | 0.0015 |
| LAVANDIN OIL | | | | 2.0000 | | | | 2.0000 |
| LAVENDER OIL | | | | | 4.0000 | | | |
| LEMONILE | | | | 1.6000 | | 0.3333 | | 1.6000 |
| LITSEA CUBEBA OIL | | | | | | | | |
| MAHONIAL | | | | | | 0.3333 | 0.5000 | |
| MANZANATE | | | | 1.0000 | | | | 1.0000 |
| MAYOL | | | 0.5 | | | | | |
| MENTHOL LAEVO | | | | | | 0.0556 | | |
| METHYL BENZOATE | | | 0.04 | | | | | |
| NECTARYL | | | 0.2 | | | | | |
| NEOCASPIRENE | | | | 0.0500 | | | | 0.0500 |
| NEROL | | 0.0400 | | | | | | |
| NEROLEX | | | | 0.5000 | | | | 0.5000 |
| NEROLIDOL | | 0.0100 | | | | | | |
| NEROLINE | | | 0.01 | | | | | |
| NONADIENOL-2,6 | | | | 0.0050 | | | | 0.0050 |
| NYMPHEAL | | | | | | 0.5556 | 1.0000 | |
| OLIBANUM OIL | | | | | | | | |
| ORANGER CRYSTALS | | | | | | | 1.0000 | |
| OXYOCTALINE FORMATE | | 0.4000 | | | | | | |
| PARMANTHEME GF | | | | | | 0.0111 | | |
| PELARGENE | | | | 0.1000 | | | | 0.1000 |
| PEONILE | | | | 10.0000 | | | | 10.0000 |
| PETALIA | | | | | | | | 8.6000 |
| PHENYL ACETALDEHYDE | | | | 0.0500 | | | | 0.0500 |
| PRUNOLIDE | | | 0.1 | | | | | |
| RASPBERRY KETONE | | | | 1.0000 | | | | 1.0000 |
| RHODINOL | | 0.2500 | | | | | | |
| RHUBAFURAN | | | 0.006 | 0.1000 | | | | 0.1000 |
| ROSACETOL | | | | 4.5000 | | | | 4.5000 |
| ROSEMARY OIL | | | | | | 0.1111 | | |
| ROSYFOLIA | | | | | | | 0.5000 | |
| SPEARMINT OIL | | | | | | 0.0111 | | |
| TETRAHYDRO LINALOOL | | | 2.8 | 8.0000 | | | | 8.0000 |
| UNDECALACTONE DELTA | | | | | | 0.0333 | | |
| UNDECATRIENE | | | | 0.0200 | | | | 0.0200 |
| VELOUTONE | | | 0.3 | | | | | |
| VERTOFIX COEUR | | | | | | 3.3333 | | |
| Sum | | 100 | 100 | 100 | 100 | 100 | 100 | 100 |

| | | | | | | | |
|---|---|---|---|---|---|---|---|
| Total % INERT | 10.24 | 34.10 | 8.96 | 1.80 | 16.14 | 33.90 | 0.36 |
| No. of R ingredients | 12 | 14 | 5 | 7 | 6 | 9 | 5 |
| Total % R ingredients (excl. INERT) | 56.27 | 45.58 | 8.91 | 54.99 | 25.84 | 29.50 | 8.14 |
| Total % NR ingredients (excl. INERT) | 14.77 | 18.23 | 25.54 | 29.74 | 11.13 | 1.51 | 23.34 |
| R/NR (excl. INERT) | 3.81 | 2.50 | 0.35 | 1.85 | 2.32 | 19.50 | 0.35 |
| Total % MISC ingredients (excl. INERT) | 17.22 | 14.10 | 50.01 | 7.13 | 20.54 | 21.26 | 54.32 |
| Total % N ingredients (excl. INERT) | 11.73 | 22.09 | 15.54 | 8.15 | 41.95 | 47.73 | 14.20 |
| R/(M + NR) (excl. INERT) | 1.76 | 1.41 | 0.12 | 1.49 | 0.82 | 1.30 | 0.10 |
| RESULT | PASS | PASS | FAIL | PASS | PASS | PASS | FAIL |

| | | H | I | J | K | L | M | N | O |
|---|---|---|---|---|---|---|---|---|---|
| Ingredient | Group | wt % | wt % | wt % | wt % | wt % | wt % | wt % | wt % |
| BENZYL BENZOATE | INERT | 0.3000 | | | | 5.0000 | | | |
| BUTYL HYDROXY TOLUENE | INERT | | | | | 0.3000 | | 0.0100 | |
| CITRIC ACID 10%/DPG | INERT | | | | | 5.0000 | | | |
| DIPROPYLENE GLYCOL | INERT | 39.8600 | 0.3571 | 27.1795 | 23.3333 | 37.6600 | 34.4400 | 21.4470 | 14.5000 |
| TRIETHYL CITRATE | INERT | | | 0.2308 | 0.2000 | | 0.0360 | | |
| BENZYL ACETATE | N | 3.5000 | | | | 3.0000 | 2.5000 | | 15.0000 |
| CASSIS BASE | N | | | 1.5385 | 1.3333 | | | | |
| CINNAMIC ALCOHOL | N | 0.2000 | | | | | | | |
| CYCLAMEN ALDEHYDE | N | | | | | | 2.0000 | | |
| DECALACTONE GAMMA | N | | 2.8571 | | | | 0.3000 | | |
| DIHYDRO EUGENOL | N | 0.2000 | | | | | | | |
| DIHYDRO MYRCENOL | N | | 7.1429 | | | | 5.0000 | 12.0000 | |
| EUGENOL | N | 0.1000 | | 0.0641 | 0.0556 | | | 0.5200 | |
| GARDENOL | N | 0.0200 | | | | | 0.5000 | | |
| HEDIONE | N | 1.0000 | 14.2857 | 12.8205 | 11.1111 | 2.0000 | 10.0000 | | |
| HYDROXYCITRONELLAL | N | | | 1.2821 | 1.1111 | | 1.0000 | 1.0000 | |
| INDOLE | N | | | 0.0256 | 0.0222 | | | | |
| ISOEUGENOL | N | | 0.0214 | | | | | | |
| JASMIN OIL | N | 0.1000 | | | | | | | |
| LINALOOL | N | 7.5000 | 7.1429 | | | 2.0000 | 7.0000 | 14.7000 | 10.0000 |
| LINALYL ACETATE | N | 0.1000 | 2.8571 | 0.2564 | 0.2222 | 2.0000 | | 9.0000 | 20.0000 |
| PATCHOULI OIL | N | | 0.0714 | 0.3846 | 0.3333 | | | 3.8000 | 0.5000 |
| PHENYL ETHYL ALCOHOL | N | 2.8000 | 0.2857 | 1.9231 | 1.6667 | | 1.0000 | | |
| ROSE OIL | N | 0.4400 | | | | | | | |
| YLANG YLANG | N | 0.2000 | | | | | 0.0300 | | |
| ALDEHYDE C 12 MNA PURE | NR | 0.2500 | | | | | | | |
| ALLYL AMYL GLYCOLATE | NR | | 0.1429 | | | | | 0.8000 | |
| AMBROFIX | NR | | | 0.1282 | 0.1111 | | 0.0500 | 0.0080 | |
| AMYL SALICYLATE | NR | | | | | | | | 7.5000 |
| ARMOISE OIL | NR | | | | | | | 0.4000 | |
| BENZYL SALICYLATE | NR | 3.0000 | 5.7143 | 19.2308 | 16.6667 | | | 15.0000 | 2.5000 |
| BERGAMOT OIL | NR | 5.0000 | | | | 4.0000 | | | |
| CITRONELLOL | NR | 2.2000 | 0.7143 | 0.6410 | 0.5556 | | 1.0000 | | 5.0000 |
| CYCLOHEXAL | NR | | 7.1429 | | | | | 1.5000 | |
| ETHYL SAFRANATE | NR | | | | | 0.0600 | | | |
| EVERNYL | NR | | | | | | | 0.4500 | 0.5000 |
| HEXENOL-3-CIS | NR | | 0.1429 | | | | 0.2500 | | |
| LEMON OIL | NR | | | | | | | 5.0000 | |
| LILIAL | NR | 4.0000 | 7.1429 | | | 3.0000 | | 0.3000 | |
| ORANGE OIL | NR | | 1.4286 | | | | 1.0000 | | |
| ORANGE TERPENES DISTILLED | NR | 1.5000 | | | | 5.6000 | | 0.2000 | |
| TRICYCLAL | NR | | | | | 0.0500 | 0.5000 | 0.0500 | |
| TROPIONAL | NR | | | | | | 1.0000 | | |
| AMBRETTOLIDE | R | | | | | | 0.1000 | | |
| AUBEPINE PARA CRESOL | R | | | | | | | | 5.0000 |
| ANETHOLE | R | | | | | | | 0.1000 | |
| BENZOIN RESINOID | R | | 0.3571 | | | 1.3000 | | | |
| COUMARIN | R | 7.5000 | 4.2857 | | | 1.8000 | | 0.5000 | |
| ETHYL VANILLIN | R | 0.5000 | 0.7143 | | | | 0.0500 | | 1.0000 |
| ETHYLENE BRASSYLATE | R | | 14.2857 | | 0.1111 | 6.0000 | 1.1000 | | |
| FLOROSA | R | | | 25.6410 | 22.2222 | | | | |
| GERANIOL | R | 1.4000 | | | | | 0.5000 | | |
| HELIOTROPINE | R | | | | | | | | 2.0000 |
| IONONE BETA | R | | | | | | 0.5000 | | |
| IRISONE ALPHA | R | | | | | | 1.0000 | | |
| ISOBUTAVAN | R | | | | | 0.0700 | | | |
| ISORALDEINE | R | 10.0000 | 5.7143 | | | | 2.5000 | | |
| METHYL ANTHRANILATE | R | | 0.1429 | | | | 0.0500 | | |
| MUSK C14 | R | | | | | 0.5000 | | | |
| PEACH PURE | R | | | | | | 1.0000 | | |
| RADJANOL | R | | 0.7143 | | | 0.2000 | | 0.4000 | |
| SERENOLIDE | R | | 1.4286 | | 13.3333 | | | | |
| SYLKOLIDE | R | 2.0000 | | | | | | | 1.0000 |
| TERPINEOL PURE | R | | 0.8571 | | | | 0.7000 | | |
| THIBETOLIDE | R | | | 1.2821 | 1.1111 | | | | |
| VANILLIN | R | | 0.7143 | | | | | | |
| VETIVER OIL | R | 0.1000 | | | | | | | |
| VELVIONE | R | | 0.1429 | | 0.1111 | | | | |
| ACETYL ISOEUGENOL | | | 0.2143 | | | | | | |
| AGRUMEX | | | | | | 4.0000 | 1.9000 | | |
| ALDEHYDE C 10 DECYLIC | | 0.1500 | 0.0286 | | | | | | 0.5000 |
| ALDEHYDE C 11 UNDECYLENIC | | 0.4000 | | | | | | | |
| ALDEHYDE C 12 LAURIC | | 0.1500 | 0.0286 | | | | | | |
| ALDEHYDE C 6 HEXYLIC | | | | | | 0.0500 | | | |
| ALLYL CAPROATE | | | | | | 0.6000 | | | |
| ALLYL CYCLOHEXYL PROPIONATE | | | | | | | 0.1500 | | |
| ALLYL OENANTHATE | | | | | | | 0.7000 | | |
| AMBRE SULTON OIL | | | 0.2857 | | | | | | |
| AMYL CINNAMIC ALDEHYDE | | 1.3000 | | | | | | | |
| ANISYL ACETATE | | | | | | 1.0000 | 0.1500 | | |
| APPLE OIL | | | | | | 2.0000 | | | |
| AURANTIOL PURE | | 0.0300 | | | | | | | |
| BENZALDEHYDE | | | 0.0286 | | | | 0.0200 | | |
| BENZYL ALCOHOL | | 0.9000 | | | | | | | |
| BUTYL ACETATE | | | | | | | 0.0100 | | |
| BUTYL CYCLOHEXYL ACETATE | | 0.4000 | | | | | | | |
| PARA | | | | | | | | | |
| CALONE | | | | | | | 0.1000 | | |
| CAMPHOR | | | | | | | | 0.1000 | |
| CASHMERAN | | | | | | | | | 0.5000 |
| CARDAMOM OIL | | | | | | | | 0.0150 | |
| CEDARWOOD OIL | | | | | | | | 1.0000 | |
| CEDRYL ACETATE | | | | | | | | 1.0000 | |
| CINNAMON BARK OIL | | | 0.0071 | | | | | 0.0100 | |
| CINNAMON LEAF OIL | | | 0.0429 | | | | | | |
| CITRAL LEMAROME N | | | 0.0714 | | | | | | |
| CITRAL | | | | | | | 0.5000 | 0.5000 | 1.0000 |
| CITRONELLYL ACETATE | | 0.4000 | 1.1429 | | | | | | |
| CLOVE BUD OIL | | | 0.0714 | | | | | | |
| CORYLONE DRIED | | | | | | 0.0300 | | | |
| CUMIN SEED OIL | | | | | | | | 0.0160 | |
| CUMINIC ALDEHYDE | | | | | | | | 0.0040 | |
| CYCLOGALBANATE | | | | | | | | 0.0600 | |
| DAMASCENONE | | | 0.0143 | 0.1026 | 0.0889 | | 0.0500 | | |
| DAMASCONE ALPHA | | | 0.0714 | | | | | | |
| DAMASCONE DELTA | | | | | | | 0.0500 | | |
| DEWFRUIT BASE | | | | | | 0.5000 | | | |
| DIETHYL MALONATE | | | | | | | 1.1000 | | |
| DIMETHYL BENZYL CARBINYL | | | | | | | 1.0000 | | |
| BUTYRATE | | | | | | | | | |
| ETHYL ACETATE | | | | | | 0.6000 | | | |
| ETHYL BUTYRATE | | | | | | | 0.1000 | | |
| ETHYL DECADIENOATE | | | | | | | 0.1000 | | |
| ETHYL HEXANOATE | | | | | | | 0.2500 | | |
| ETHYL MALTOL | | | 0.0714 | | | 3.0000 | 0.1000 | | |
| ETHYL METHYL-2-BUTYRATE | | | | | | | 0.2500 | | |
| EUCALYPTOL | | | 0.1429 | | | | | | |
| FLORALOZONE | | | | 0.7692 | 0.6667 | | 1.0000 | | |
| FLORHYDRAL | | | | | | | 0.2000 | | |
| FLOROCYCLENE | | | | | | | 1.0000 | | |
| GARDOCYCLENE | | 0.2000 | | | | | | | |
| GERANIUM OIL | | 1.2000 | | | | | | | 5.0000 |
| GERANODYLE | | | | | | | | | 2.5000 |
| GERANYL ACETATE | | | | 0.2564 | 0.2222 | | 1.5000 | | |
| HEXENYL-3-CIS ACETATE | | | | | | 0.1000 | 0.2000 | | |
| HEXENYL-3-CIS SALICYLATE | | | | | | | 1.2000 | | |
| HEXYL ACETATE | | | | | | 2.5000 | 1.0500 | | |
| HEXYL CINNAMIC ALDEHYDE | | | 10.7143 | | | 5.0000 | 0.5000 | | |
| ISO E SUPER | | | | 3.8462 | 3.3333 | | 4.5000 | | |
| ISOAMYL ACETATE | | | | | | | 1.4000 | | |
| ISOCYCLOCITRAL | | | | | | | 0.0500 | | |
| JUNIPER OIL | | | | | | | | 0.1600 | |
| LAVANDIN OIL | | | | | | | | 5.0000 | 5.0000 |
| LIFFAROME | | | | | | | 0.0200 | | |
| LINALYL ISOBUTYRATE | | | | | | 0.5000 | | | |
| MAGNOLAN | | | | | | | 1.5000 | | |
| MALTYL ISOBUTYRATE | | | | | | 0.0600 | | | |
| MANZANATE | | | | | | 0.3000 | 0.0500 | | |
| MELONAL | | | | | | | 0.0700 | | |
| METHOXYMELONAL | | | | | | | 0.0600 | | |
| METHYL CINNAMATE | | | 0.0714 | | | | 0.0200 | | |
| METHYL ISOEUGENOL | | | | 0.0256 | 0.0222 | | | | |
| METHYL OCTYNE CARBONATE | | | | | | | 0.0100 | | |
| METHYL PAMPLEMOUSSE | | | | 1.2821 | 1.1111 | | | | |
| NEOFOLIONE | | | | | | | 0.1000 | | |
| NEROLI 27/20 | | 0.2000 | | | | | | | |
| NONADIENYL ACETATE | | | | | | | 0.0040 | | |
| NYMPHEAL | | | | 0.3846 | 0.3333 | | 0.7000 | | 1.0000 |
| PHENYL ETHYL ACETATE | | 0.2000 | | 0.1154 | 0.1000 | | 0.5000 | | |
| PINOACETALDEHYDE | | | | | | | 0.1000 | | |
| PRENYL ACETATE | | | | | | 0.0500 | 0.0500 | | |
| PRUNOLIDE | | | | 0.1282 | 0.1111 | | 0.2000 | | |
| RASPBERRY KETONE | | | | 0.3846 | 0.3333 | | 0.4000 | | |
| ROSACETOL | | 0.7000 | | | | | | | |
| ROSEMARY OIL | | | | | | | | 4.5000 | |
| ROSYFOLIA | | | | | | | 0.1000 | | |
| SCHINUS MOLLE OIL | | | | 0.0256 | 0.0222 | | | | |
| SPEARMINT OIL | | | 0.0143 | | | | | | |
| STRAWBERRY OLIFFAC | | | | | | 0.1000 | | | |
| STRAWBERRY PURE | | | | 0.0513 | 0.0444 | 0.0700 | 0.0300 | | |
| STYRAX RESINOID | | | | | | | | 0.4500 | |
| THYMOL | | | 0.0286 | | | | | | |
| UNDECAVERTOL | | | | | | | 2.0000 | | |
| VETIKOL ACETATE/CORPS | | | | | | | 0.1000 | | |
| RHUBARB | | | | | | | | | |
| Sum | | 100 | 100 | 100 | 100 | 100 | 100 | 100 | 100 |

| Total % INERT | 40.16 | 0.36 | 27.41 | 23.53 | 47.96 | 34.48 | 21.46 | 14.50 |
|---|---|---|---|---|---|---|---|---|
| No. of R ingredients | 6 | 11 | 2 | 5 | 6 | 10 | 3 | 4 |
| Total % R ingredients (excl. INERT) | 35.93 | 29.46 | 37.09 | 48.24 | 18.97 | 11.45 | 1.27 | 10.53 |
| Total % NR ingredients (excl. INERT) | 26.65 | 22.51 | 27.55 | 22.67 | 24.42 | 5.80 | 30.18 | 18.13 |
| R/NR (excl. INERT) | 1.35 | 1.31 | 1.35 | 2.13 | 0.78 | 1.97 | 0.04 | 0.58 |
| Total % MISC ingredients (excl. INERT) | 10.41 | 13.10 | 10.16 | 8.36 | 39.32 | 38.37 | 16.32 | 18.13 |
| Total % N ingredients (excl. INERT) | 27.01 | 34.79 | 25.20 | 20.74 | 17.29 | 44.76 | 52.23 | 53.22 |
| R/(M + NR) (excl. INERT) | 0.97 | 0.83 | 0.98 | 1.56 | 0.30 | 0.26 | 0.03 | 0.29 |
| RESULT | PASS | PASS | FAIL | PASS | FAIL | FAIL | FAIL | FAIL |

Compositions A through O were subjected to fNIRS and/or Mood Portraits® testing. Among these, Compositions C, J, L, M, and N are comparative examples; all other compositions are fragrance compositions according to the present invention.

Ingredients contained in these compositions are specified in the two tables above.

### EXAMPLE 4: RESULTS OF FUNCTIONAL NEAR INFRARED SPECTROSCOPY TESTING

fNIRS testing of fragrance compositions A through O described in Example 3 was conducted according to the method described in Example 1. A non-odour control was used as the benchmark.

As a first level, the following six conditions were investigated:
Condition 1: Deoxy Hb for the right brain hemisphere shows a statistically significant increase after 5-10 seconds of smelling;
Condition 2: Oxy Hb for the full brain shows a statistically significant decrease after 30 seconds of smelling;
Condition 3: Oxy Hb for the right brain hemisphere shows a statistically significant decrease after 10 seconds of smelling;
Condition 4: Oxy Hb for the right brain hemisphere shows a statistically significant decrease after 0-5 seconds of smelling;
Condition 5: Oxy Hb for the full brain shows a statistically significant decrease after 0-5 seconds of smelling; and
Condition 6: Oxy Hb for the right brain hemisphere shows a statistically significant decrease after 30 seconds of smelling.

Based on extensive testing, it had been determined that at least three of the above Conditions 1 through 6 are met in case a fragrance composition is relaxing.

The results of the first level fNIRS testing are shown in the following two tables:

| | | A | B | C | D | E | F | G |
|---|---|---|---|---|---|---|---|---|
| Condition 1 | Test Fragrance | 0.016 | −0.042 | 0.041 | 0.019 | −0.037 | 0.125 | 0.023 |
| | Benchmark | −0.181 | −0.181 | −0.002 | 0.025 | 0.025 | 0.046 | −0.002 |
| | Condition met | YES | YES | YES | NO | NO | YES | YES |
| Condition 2 | Test Fragrance | 0.012 | −0.059 | 0.008 | −0.039 | −0.100 | −0.001 | 0.027 |
| | Benchmark | 0.131 | 0.131 | 0.010 | 0.080 | 0.080 | 0.004 | 0.010 |
| | Condition met | YES | YES | NO | YES | YES | NO | NO |
| Condition 3 | Test Fragrance | −0.039 | −0.034 | 0.016 | −0.127 | −0.173 | −0.140 | −0.121 |
| | Benchmark | 0.186 | 0.186 | −0.074 | −0.064 | −0.064 | −0.124 | −0.074 |
| | Condition met | YES | YES | NO | YES | YES | YES | YES |
| Condition 4 | Test Fragrance | −0.018 | −0.170 | 0.023 | −0.150 | −0.067 | −0.089 | 0.099 |
| | Benchmark | 0.233 | 0.233 | −0.059 | −0.098 | −0.098 | −0.098 | −0.059 |
| | Condition met | YES | YES | NO | YES | NO | NO | YES |
| Condition 5 | Test Fragrance | −0.126 | −0.114 | −0.046 | −0.206 | −0.062 | −0.041 | −0.082 |
| | Benchmark | 0.109 | 0.109 | −0.018 | −0.094 | −0.094 | −0.106 | −0.018 |
| | Condition met | YES | YES | NO | YES | NO | NO | YES |
| Condition 6 | Test Fragrance | 0.062 | −0.107 | 0.032 | 0.018 | −0.074 | −0.018 | 0.059 |
| | Benchmark | 0.195 | 0.195 | −0.017 | 0.075 | 0.075 | 0.021 | −0.017 |
| | Condition met | YES | YES | NC | YES | YES | YES | YES |

| | | | | | | | |
|---|---|---|---|---|---|---|---|
| Number of conditions met | 6 | 6 | 1 | 5 | 3 | 3 | 5 |
| Relaxing effect? | YES | YES | NO | YES | YES | YES | YES |

| | | H | I | J | K | L | M | N | |
|---|---|---|---|---|---|---|---|---|---|
| Condition 1 | Test Fragrance | 0.122 | 0.106 | 0.208 | 0.010 | −0.025 | 0.036 | 0.115 | 0.052 |
| | Benchmark | 0.016 | −0.033 | −0.029 | −0.029 | 0.006 | −0.018 | 0.025 | 0.081 |
| | Condition met | YES | YES | YES | YES | NO | YES | YES | NO |
| Condition 2 | Test Fragrance | −0.061 | −0.077 | 0.009 | 0.052 | 0.014 | 0.023 | 0.102 | −0.118 |
| | Benchmark | 0.049 | 0.066 | −0.079 | −0.079 | 0.006 | −0.097 | 0.080 | 0.098 |
| | Condition met | YES | YES | NO | NO | NO | NO | NO | YES |
| Condition 3 | Test Fragrance | −0.109 | −0.161 | −0.084 | −0.098 | 0.116 | −0.031 | −0.055 | −0.145 |
| | Benchmark | −0.027 | −0.067 | −0.119 | −0.119 | −0.002 | −0.090 | −0.064 | −0.007 |
| | Condition met | YES | YES | NO | NO | NO | NO | NO | YES |
| Condition 4 | Test Fragrance | −0.084 | −0.186 | 0.041 | −0.097 | 0.193 | 0.007 | −0.111 | −0.176 |
| | Benchmark | 0.018 | −0.127 | −0.027 | −0.027 | −0.025 | −0.139 | −0.098 | 0.034 |
| | Condition met | YES | YES | NO | YES | NO | NO | NO | YES |
| Condition 5 | Test Fragrance | −0.118 | −0.197 | −0.010 | −0.102 | 0.136 | 0.032 | 0.100 | −0.210 |
| | Benchmark | 0.007 | −0.125 | −0.052 | −0.052 | −0.032 | −0.096 | −0.094 | −0.001 |
| | Condition met | YES | YES | NO | YES | NO | NO | NO | YES |
| Condition 6 | Test Fragrance | −0.072 | −0.062 | 0.012 | 0.031 | 0.029 | 0.005 | 0.100 | −0.104 |
| | Benchmark | 0.051 | 0.079 | −0.091 | −0.091 | 0.012 | −0.123 | 0.075 | 0.113 |
| | Condition met | YES | YES | NO | NO | NO | NO | NO | YES |

| | | | | | | | | |
|---|---|---|---|---|---|---|---|---|
| Number of conditions met | 6 | 6 | 1 | 3 | 0 | 1 | 1 | 5 |
| Relaxing effect? | YES | YES | NO | YES | NO | NO | NO | YES |

For those compositions that fulfilled the first level fNIRS requirements (Compositions A, B, D, E, F, G, H, I, K, and O), a further investigation of specific fNIRS channels and time points was conducted. Specifically, it was tested if one or more of the following conditions were met:
C1. Channel 6: statistically significant increase of Deoxy Hb after 30 seconds of smelling;
C2. Channel 15: statistically significant increase of Deoxy Hb after 30 seconds of smelling;
C3. Channel 3: statistically significant increase of Deoxy Hb after 0-5 seconds of smelling;
C4. Channel 5: statistically significant increase of Deoxy Hb after 0-5 seconds of smelling;
C5. Channel 8: statistically significant decrease of Deoxy Hb after 0-5 seconds of smelling;
C6. Channel 15: statistically significant increase of Deoxy Hb after 0-5 seconds of smelling;
C7. Channel 8: statistically significant decrease of Deoxy Hb after 0-10 seconds of smelling;
C8. Channel 15: statistically significant increase of Deoxy Hb after 0-10 seconds of smelling;
C9. Channel 5: statistically significant increase of Deoxy Hb after 5-10 seconds of smelling;
C10. Channel 8: statistically significant decrease of Deoxy Hb after 5-10 seconds of smelling;
C11. Channel 15: statistically significant increase of Deoxy Hb after 5-10 seconds of smelling;
C12. Channel 20: statistically significant decrease of Total Hb after 30 seconds of smelling;
C13. Channel 10: statistically significant decrease of Total Hb after 0-5 seconds of smelling;
C14. Channel 12: statistically significant decrease of Total Hb after 0-10 seconds of smelling;
C15. Channel 4: statistically significant increase of Oxy Hb after 30 seconds of smelling;
C16. Channel 17: statistically significant decrease of Oxy Hb after 30 seconds of smelling;
C17. Channel 15: statistically significant decrease of Oxy Hb after 0-5 seconds of smelling;
C18. Channel 3: statistically significant decrease of Oxy Hb after 5-10 seconds of smelling;
C19. Channel 16: statistically significant decrease of Oxy Hb after 5-10 seconds of smelling;
C20. Channel 20: statistically significant decrease of Oxy Hb after 5-10 seconds of smelling.

Preferred relaxing fragrance compositions were found to meet at least ten of the above conditions C1 through C20 (level 2).

Particularly preferred compositions were found to meet at least five of conditions C2, C4, C5, C6, C9, C11, C13, C15, C16, and C20.

The results of the second and third level fNIRS testing are shown in the table below.

As can be seen from this table, most tested fragrance compositions that meet the requirements of the first level fNIRS testing also meet those of the second and third level. The exceptions are Composition O, which meets the first and second level but not the third, and Composition G, which only meets the first level.

Thus, the compositions of the present invention were found to be relaxing on the sub-conscious level.

| | | A | B | D | E | F | G | H | I | K | O |
|---|---|---|---|---|---|---|---|---|---|---|---|
| C1 | Test Fragrance | 0.194 | −0.164 | −0.081 | 0.257 | 0.036 | −0.147 | −0.093 | 0.161 | −0.158 | 0.014 |
| | Benchmark | 0.011 | 0.011 | −0.075 | −0.075 | −0.025 | 0.179 | 0.044 | 0.063 | 0.033 | 0.012 |
| | Condition met | YES | NO | NO | YES | YES | YES | NO | YES | NO | NO |
| C2 | Test Fragrance | −0.024 | 0.056 | 0.196 | 0.117 | 0.117 | −0.080 | 0.117 | 0.038 | 0.077 | −0.085 |
| | Benchmark | −0.052 | −0.052 | −0.323 | −0.323 | −0.018 | 0.067 | −0.017 | −0.038 | −0.043 | −0.085 |
| | Condition met | NO | YES | YES | NO | YES | NO | YES | YES | YES | NO |
| C3 | Test Fragrance | 0.100 | 0.073 | 0.118 | 0.108 | 0.267 | 0.035 | 0.112 | 0.294 | 0.174 | 0.102 |
| | Benchmark | 0.066 | 0.066 | −0.059 | −0.059 | 0.435 | 0.141 | 0.250 | 0.208 | 0.268 | 0.163 |
| | Condition met | NO | NO | YES | YES | NO | NO | NO | YES | NO | NO |
| C4 | Test Fragrance | 0.124 | 0.074 | −0.051 | 0.166 | −0.007 | 0.023 | 0.413 | 0.135 | 0.154 | −0.146 |
| | Benchmark | −0.111 | −0.111 | −0.102 | −0.102 | 0.190 | −0.010 | −0.196 | 0.195 | −0.084 | 0.277 |
| | Condition met | YES | YES | YES | YES | NO | NO | YES | NO | YES | NO |
| C5 | Test Fragrance | −0.161 | −0.118 | 0.022 | 0.088 | −0.191 | −0.126 | 0.109 | −0.077 | 0.055 | −0.027 |
| | Benchmark | 0.095 | 0.095 | 0.048 | 0.048 | 0.094 | −0.199 | −0.071 | 0.148 | 0.202 | 0.121 |
| | Condition met | YES | YES | YES | NO | YES | NO | NO | YES | YES | YES |
| C6 | Test Fragrance | −0.148 | −0.047 | 0.159 | −0.090 | −0.108 | −0.054 | 0.261 | 0.068 | 0.126 | −0.085 |
| | Benchmark | −0.332 | −0.332 | −0.411 | −0.411 | −0.071 | 0.043 | 0.036 | 0.019 | −0.052 | 0.133 |
| | Condition met | YES | YES | YES | YES | NO | NO | YES | YES | YES | NO |
| C7 | Test Fragrance | −0.139 | −0.200 | 0.054 | 0.082 | −0.114 | −0.081 | 0.072 | −0.040 | −0.004 | 0.003 |
| | Benchmark | 0.083 | 0.083 | 0.105 | 0.105 | 0.022 | −0.191 | −0.077 | 0.120 | 0.197 | 0.056 |
| | Condition met | YES | YES | YES | YES | YES | NO | NO | YES | YES | YES |
| C8 | Test Fragrance | −0.016 | −0.006 | 0.230 | −0.004 | 0.037 | −0.011 | 0.226 | 0.091 | 0.117 | −0.044 |
| | Benchmark | −0.265 | −0.265 | −0.372 | −0.372 | −0.068 | 0.121 | 0.051 | 0.019 | −0.120 | 0.083 |
| | Condition met | YES | YES | YES | YES | YES | NO | YES | YES | YES | NO |
| C9 | Test Fragrance | 0.025 | −0.158 | 0.091 | 0.243 | 0.098 | −0.053 | 0.256 | 0.088 | 0.163 | −0.249 |
| | Benchmark | −0.202 | −0.202 | −0.046 | −0.046 | 0.191 | −0.077 | −0.275 | 0.018 | −0.134 | 0.106 |
| | Condition met | YES | YES | YES | NO | NO | NO | YES | YES | YES | NO |
| C10 | Test Fragrance | −0.117 | −0.282 | 0.086 | 0.076 | −0.037 | −0.037 | 0.035 | −0.004 | −0.063 | 0.032 |
| | Benchmark | 0.071 | 0.071 | 0.163 | 0.163 | −0.050 | −0.183 | −0.083 | 0.091 | 0.192 | −0.009 |
| | Condition met | YES | YES | YES | YES | NO | NO | NO | YES | YES | NO |
| C11 | Test Fragrance | 0.116 | 0.034 | 0.302 | 0.081 | 0.183 | 0.033 | 0.190 | 0.115 | 0.107 | −0.003 |
| | Benchmark | −0.198 | −0.198 | −0.333 | −0.333 | −0.066 | 0.199 | 0.066 | 0.019 | −0.187 | 0.033 |
| | Condition met | YES | YES | YES | YES | YES | NO | YES | YES | YES | NO |
| C12 | Test Fragrance | 0.1116 | −0.190 | 0.110 | −0.244 | 0.019 | 0.112 | −0.048 | 0.025 | 0.077 | −0.073 |
| | Benchmark | 0.143 | 0.143 | 0.132 | 0.132 | 0.011 | −0.037 | 0.049 | 0.054 | −0.032 | 0.011 |
| | Condition met | NO | YES | NO | YES | NO | NO | YES | NO | NO | YES |
| C13 | Test Fragrance | −0.289 | −0.157 | −0.395 | −0.406 | −0.021 | −0.131 | −0.113 | −0.310 | −0.197 | −0.248 |
| | Benchmark | −0.013 | −0.013 | −0.356 | −0.356 | −0.346 | −0.029 | −0.114 | −0.250 | −0.086 | −0.070 |
| | Condition met | YES | YES | NO | YES | NO | YES | NO | YES | YES | YES |
| C14 | Test Fragrance | −0.145 | −0.285 | −0.059 | 0.177 | −0.126 | −0.200 | −0.075 | −0.224 | 0.080 | −0.385 |
| | Benchmark | 0.154 | 0.154 | −0.155 | −0.155 | −0.133 | −0.101 | −0.234 | 0.058 | −0.065 | −0.128 |
| | Condition met | YES | YES | YES | NO | NO | YES | NO | YES | NO | YES |
| C15 | Test Fragrance | 0.101 | 0.107 | −0.082 | −0.058 | 0.008 | −0.022 | −0.121 | −0.218 | 0.129 | −0.172 |
| | Benchmark | −0.199 | −0.199 | 0.135 | 0.135 | −0.011 | 0.020 | 0.015 | 0.002 | −0.033 | 0.116 |
| | Condition met | YES | YES | NO | NO | YES | NO | NO | NO | YES | NO |
| C16 | Test Fragrance | −0.044 | −0.042 | 0.135 | −0.001 | −0.044 | 0.017 | −0.142 | 0.021 | 0.022 | −0.068 |
| | Benchmark | 0.186 | 0.186 | 0.051 | 0.051 | 0.044 | 0.077 | 0.183 | 0.076 | −0.092 | 0.214 |
| | Condition met | YES | YES | NO | YES | YES | YES | YES | YES | NO | YES |
| C17 | Test Fragrance | −0.202 | −0.250 | −0.321 | −0.225 | −0.169 | −0.072 | −0.147 | −0.277 | −0.218 | −0.386 |
| | Benchmark | 0.099 | 0.099 | −0.213 | −0.213 | −0.199 | −0.319 | −0.102 | −0.313 | −0.030 | −0.066 |
| | Condition met | YES | YES | YES | NO | NO | NO | YES | NO | YES | YES |
| C18 | Test Fragrance | −0.185 | 0.054 | −0.248 | 0.095 | −0.331 | −0.208 | 0.004 | 0.213 | 0.143 | −0.267 |
| | Benchmark | 0.019 | 0.019 | 0.354 | 0.354 | −0.082 | 0.094 | 0.193 | 0.050 | 0.066 | 0.057 |
| | Condition met | YES | NO | YES | YES | YES | YES | YES | NO | NO | YES |
| C19 | Test Fragrance | −0.091 | −0.209 | −0.091 | −0.008 | −0.225 | 0.021 | −0.154 | −0.203 | 0.126 | −0.175 |
| | Benchmark | 0.291 | 0.291 | 0.054 | 0.054 | −0.152 | −0.073 | −0.051 | −0.010 | −0.125 | −0.008 |
| | Condition met | YES | YES | YES | YES | YES | NO | YES | YES | NC | YES |
| C20 | Test Fragrance | 0.072 | −0.034 | 0.114 | −0.136 | 0.084 | 0.119 | 0.025 | 0.039 | 0.104 | −0.213 |
| | Benchmark | 0.320 | 0.320 | 0.335 | 0.335 | 0.042 | −0.012 | 0.192 | 0.253 | −0.046 | 0.011 |
| | Condition met | YES | YES | YES | YES | NO | NO | YES | YES | NO | YES |

| | | | | | | | | | | |
|---|---|---|---|---|---|---|---|---|---|---|
| Number of conditions met among | 17 | 17 | 15 | 14 | 10 | 5 | 12 | 15 | 12 | 10 |
| C1-C20 (level 2) | | | | | | | | | | |
| Relaxing effect (level 2) | YES | YES | YES | YES | YES | NO | YES | YES | YES | YES |
| Number of conditions met among | 9 | 10 | 7 | 6 | 5 | 2 | 7 | 8 | 8 | 4 |
| C2, C4, C5, C6, C9, C11, C13, C15, | | | | | | | | | | |
| C16, and C20 (level 3) | | | | | | | | | | |
| Relaxing effect (level 3) | YES | YES | YES | YES | YES | NO | YES | YES | YES | NO |

### EXAMPLE 5: RESULTS FROM QUESTIONNAIRE

The following table shows an example for results obtained with the questionnaire used in Example 2 (14 participants). Composition H of Example 3 is the test fragrance; a non-odour control was used as the benchmark; and Fragrance 1 and 2 are comparative examples included to complete the set of 4.

| | Liking | Strength | Invigorating | Relaxing |
|---|---|---|---|---|
| Sample | (1-9) | (1-9) | (1-9) | (1-9) |
| Fragrance 1 | 7.214 | 6.500 | 6.357 | 4.214 |
| Test | 6.786 | 6.286 | 5.643 | 6.286 |
| Fragrance 2 | 6.357 | 6.286 | 4.714 | 5.214 |
| Benchmark | 4.714 | 1.643 | 1.357 | 1.786 |
| (non-odour) | | | | |

Thus, also on the conscious level, Composition H was found to be relaxing.

### EXAMPLE 6: RESULTS OF MOOD PORTRAITS® TESTING

In addition to the fNIRS testing, a Mood Portraits® study as described in Example 2 was also conducted on a large number of fragrance compositions.

For the present invention, the results of the Mood Portraits® study were analysed with regard to a relaxed mood. Specifically, the selection frequency of pictures associated with relaxation and the grade of association of the respective pictures with a relaxed mood (some pictures are very strongly associated with relaxation, whereas it is only one association among several equally strong ones for other pictures) were taken into account.

A comparison of several dozen fragrance compositions showed that most of them have a very similar effect on relaxation; but a few fragrance compositions are able to significantly evoke or not evoke a relaxed mood.

FIG. 2 shows the results for some of the fragrance compositions that were tested, namely for Compositions A (according to the invention) and M (comparative example) of Example 3, and of Compositions P through W, which were not previously described in this disclosure.

More precisely, FIG. 2 shows the odds ratio for a relaxed mood, indicating for each fragrance composition if it evokes a relaxed mood more or less than the other compositions. The odds ratios (horizontal lines) are shown with 95% confidence intervals (vertical lines). If the 95% confidence interval for a fragrance composition is entirely above the Significance Line of 1.0, then said fragrance composition significantly evokes a more relaxed mood; if the 95% confidence interval for a fragrance composition is entirely below the Significance Line of 1.0, then said fragrance composition significantly evokes a less relaxed mood.

Thus, as can be seen from FIG. 2, Composition A, which is a fragrance composition according to the present invention, is able to evoke a relaxed mood significantly more than all the other fragrance compositions. Composition M, on the other hand, evokes a significantly less relaxed mood than the others. Finally, Compositions P through W essentially lie on the Significance Line.

Thus, the Mood Portraits® results confirm that Composition A, which has been found to be relaxing in the fNIRS study and which also complies with the formulation guidelines of the present invention, significantly more evokes relaxation compared to a large majority of other fragrance compositions.

1. A method of assessing the ability of a test fragrance ingredient or a test fragrance composition to improve the relaxation state of a human subject, comprising the steps of:
a) measuring a base relaxation state of one or more human test subject(s);
b) providing the test fragrance ingredient or the test fragrance composition to the human test subject(s) for smelling;
c) measuring a resulting relaxation state of the human test subject(s); and
d) determining a difference between the resulting relaxation state and the base relaxation state for the human test subject(s);
wherein the base relaxation state and the resulting relaxation state are measured by functional Near Infrared Spectroscopy (fNIRS) of the human test subject(s)' left brain hemisphere, right brain hemisphere, and full brain;
wherein the test fragrance ingredient or the test fragrance composition is able to improve the relaxation state of the human subject if at least three out of the following six conditions are met:
Condition 1: Deoxy Hb for the right brain hemisphere shows a statistically significant increase after 5-10 seconds of smelling;
Condition 2: Oxy Hb for the full brain shows a statistically significant decrease after 30 seconds of smelling;
Condition 3: Oxy Hb for the right brain hemisphere shows a statistically significant decrease after 0-10 seconds of smelling;
Condition 4: Oxy Hb for the right brain hemisphere shows a statistically significant decrease after 0-5 seconds of smelling;
Condition 5: Oxy Hb for the full brain shows a statistically significant decrease after 0-5 seconds of smelling;
Condition 6: Oxy Hb for the right brain hemisphere shows a statistically significant decrease after 30 seconds of smelling;
wherein Deoxy Hb is the amount of deoxygenated haemoglobin measured and Oxy Hb is the amount of oxygenated haemoglobin measured.

2. The method of claim 1, wherein at least four out of the Conditions 1-6 are met, more preferably at least five, and most preferably all six.

3. The method of claim 1, wherein at least 10, more preferably at least 12, and most preferably at least 15, of the following conditions are met:
C1. Channel 6 shows a statistically significant increase of Deoxy Hb after 30 seconds of smelling;
C2. Channel 15 shows a statistically significant increase of Deoxy Hb after 30 seconds of smelling;
C3. Channel 3 shows a statistically significant increase of Deoxy Hb after 0-5 seconds of smelling;
C4. Channel 5 shows a statistically significant increase of Deoxy Hb after 0-5 seconds of smelling;
C5. Channel 8 shows a statistically significant decrease of Deoxy Hb after 0-5 seconds of smelling;
C6. Channel 15 shows a statistically significant increase of Deoxy Hb after 0-5 seconds of smelling;
C7. Channel 8 shows a statistically significant decrease of Deoxy Hb after 0-10 seconds of smelling;
C8. Channel 15 shows a statistically significant increase of Deoxy Hb after 0-10 seconds of smelling;
C9. Channel 5 shows a statistically significant increase of Deoxy Hb after 5-10 seconds of smelling;
C10. Channel 8 shows a statistically significant decrease of Deoxy Hb after 5-10 seconds of smelling;
C11. Channel 15 shows a statistically significant increase of Deoxy Hb after 5-10 seconds of smelling;
C12. Channel 20 shows a statistically significant decrease of Total Hb after 30 seconds of smelling;
C13. Channel 10 shows a statistically significant decrease of Total Hb after 0-5 seconds of smelling;
C14. Channel 12 shows a statistically significant decrease of Total Hb after 0-10 seconds of smelling;
C15. Channel 4 shows a statistically significant increase of Oxy Hb after 30 seconds of smelling;
C16. Channel 17 shows a statistically significant decrease of Oxy Hb after 30 seconds of smelling;
C17. Channel 15 shows a statistically significant decrease of Oxy Hb after 0-5 seconds of smelling;
C18. Channel 3 shows a statistically significant decrease of Oxy Hb after 5-10 seconds of smelling;
C19. Channel 16 shows a statistically significant decrease of Oxy Hb after 5-10 seconds of smelling;
C20. Channel 20 shows a statistically significant decrease of Oxy Hb after 5-10 seconds of smelling;
wherein Channels 1 to 8 and 11 are located in the left brain hemisphere, Channels 9 and 12 are located on the midline, and Channels 10 and 13 to 20 are located in the right brain hemisphere, and wherein Total Hb is the amount of total haemoglobin measured.

4. The method of claim 3, wherein at least 5, preferably at least 6, more preferably at least 7, even more preferably at least 8, still more preferably at least 9, and most preferably all 10, of the following conditions are met: C2; C4; C5; C6; C9; C11; C13; C15; C16; and C20.

5. A method of creating a fragrance composition having a relaxing effect on a human subject, comprising the steps of:
(i) creating a test fragrance composition;
(ii) assessing the ability of the test fragrance composition to improve the relaxation state of a human subject according to the method of claim 1, and
(iii) adjusting, if necessary, the test fragrance composition by adding and/or removing at least one fragrance ingredient and/or increasing and/or reducing the concentration of at least one fragrance ingredient until the fragrance composition is found to improve the relaxation state of the human subject.

6. The method of claim 5, wherein, in step (iii), at least one relaxing fragrance material R is added to the test fragrance composition and/or at least one non-relaxing fragrance material NR is removed from the test fragrance composition and/or the concentration of at least one relaxing fragrance material R is increased and/or the concentration of at least one non-relaxing fragrance material NR is reduced, wherein
the relaxing fragrance materials R are selected from the group consisting of a musk ingredient, a floral-orris ingredient, a sweet-vanilla ingredient, a sweet-heliotrope ingredient, a sweet-tonka ingredient, a woody-vetiver ingredient, 2-(phenoxy)ethyl 2-methylpropanoate (phenoxyethyl isobutyrate), 1-(2-naphtalenyl)-ethanone (oranger crystals), 2-(4-methylcyclohex-3-en-1-yl)propan-2-ol (terpineol), (E)-1-methoxy-4-(prop-1-en-1-yl)benzene (anethole) 2-ethyl-4(2′,2′,3′-trimethylcyclopent-3-enyl)but-enol (Bangalol or Radjanol), basil oil, (E)-3,7-dimethylnona-1,6-dien-3-ol (ethyl linalool), 2-(2′-methylpropyl)-4-hydroxy-4-methyltetrahydropyran (Florosa), (E)-3,7-dimethyl-octa-2,6-dien-1-ol (geraniol), methyl 2-aminobenzoate (methyl anthranilate), mixtures of 2-methyl-1-phenylpropan-2-yl butanoate and (phenoxy)ethyl 2-methylpropanoate (Prunella), 5-heptyldihydrofuran-2(3H)-one (gamma undecalactone or peach pure), and mixtures thereof; and
the non-relaxing fragrance materials NR are selected from the group consisting of 2-methylundecanal (methyl nonyl aldehyde), prop-2-enyl 2-(3-methylbutoxy)acetate (allyl amyl glycolate), (Z)-1-((3aS,6R,7R,8aS)-6,8,8-trimethyloctahydro-3H-3a,7-methano-azulen-3-ylidene)propan-2-one (acetyl cedrene), 3a,6,6,9a-perhydrotetramethyl-naphtho [2,1-b] furan (Amberlyn Super), pentyl 2-hydroxybenzoate (amyl salicylate), armoise oil, benzyl 2-hydroxybenzoate (benzyl salicylate), bergamot oil, 4-tert-butyl-3-phenylpropanal (Bourgeonal), cedar leaf oil, 3,7-dimethyloct-6-en-1-ol (citronellol), (E)-1-(2,6,6-trimethyl-1-cyclohexenyl)but-2-en-1-one (beta damascene), 2-methyl-1-phenylpropan-2-yl acetate (dimethyl benzyl carbinyl acetate), ethyl 2,6,6-trimethylcyclohexadienecarboxylate (Ethyl Safranate), methyl 2,4-dihydroxy-3,6-dimethylbenzoate (Evernyl or Everniate), (E)-3,7-dimethylocta-2,6-dienenitrile (geranyl nitrile), 3-(1,3-benzodioxol-5-yl)-2-methylpropanal (Helional or Tropional), Z-hex-3-enol, hexyl 2-hydroxybenzoate (hexyl salicylate), lemon oil, 2,4-dimethyl-3-cyclohexene-1-carbaldehyde (Cyclal C or Tricyclal or Ligustral), 3-(4-(1,1-dimethylethyl)phenyl-2-methylpropanal (Lilial), 4-(4-hydroxy-4-methylpentyl)cyclohex-3-enecarbaldehyde (Lyral), 3-methyl-5-phenylpentanol (Mefrosol), orange oil, orange terpenes, tagetes oil, 3,7-dimethyloctan-1-ol (pelargol), and mixtures thereof.

7. A fragrance composition for improving the relaxation state of a human subject, the fragrance composition comprising:
a) at least about 25% by weight in total of at least five relaxing fragrance materials R;
b) optionally up to about 45% by weight in total of non-relaxing fragrance materials NR, provided that the weight ratio of R to NR is at least 0.75;
c) optionally up to about 75% by weight in total of neutral fragrance materials N; and
d) optionally up to about 25% by weight in total of other fragrance materials M, provided that the weight ratio of R to (M+NR) is at least 0.75;
wherein
(i) all percentages are based on total weight of the fragrance materials constituting the fragrance composition;
(ii) the relaxing fragrance materials R are selected from the group consisting of a musk ingredient, a floral-orris ingredient, a sweet-vanilla ingredient, a sweet-heliotrope ingredient, a sweet-tonka ingredient, a woody-vetiver ingredient, 2-(phenoxy)ethyl 2-methylpropanoate (phenoxyethyl isobutyrate), 1-(2-naphtalenyl)-ethanone (oranger crystals), 2-(4-methylcyclohex-3-en-1-yl)propan-2-ol (terpineol), (E)-1-methoxy-4-(prop-1-en-1-yl)benzene (anethole) 2-ethyl-4(2′,2′,3′-trimethylcyclopent-3-enyl)but-enol (Bangalol or Radjanol), basil oil, (E)-3,7-dimethylnona-1,6-dien-3-ol (ethyl linalool), 2-(2′-methylpropyl)-4-hydroxy-4-methyltetrahydropyran (Florosa), (E)-3,7-dimethylocta-2,6-dien-1-ol (geraniol), methyl 2-aminobenzoate (methyl anthranilate), (E)-3-methyl-4-(2,6,6-trimethyl-1-cyclohex-2-enyl)but-3-en-2-one (alpha-iso-methyl ionone), mixtures of 2-methyl-1-phenylpropan-2-yl butanoate and (phenoxy)ethyl 2-methylpropanoate (Prunella), 5-heptyldihydrofuran-2(3H)-one (gamma undecalactone or peach pure), and mixtures thereof;
(iii) the non-relaxing fragrance materials NR are selected from the group consisting of 2-methylundecanal (methyl nonyl aldehyde), prop-2-enyl 2-(3-methylbutoxy)acetate (allyl amyl glycolate), (Z)-1-((3aS,6R,7R,8aS)-6,8,8-trimethyloctahydro-3H-3a,7-methanoazulen-3-ylidene)propan-2-one (acetyl cedrene), 3a,6,6,9a-perhydrotetra-methylnaphtho [2,1-b] furan (Amberlyn Super), pentyl 2-hydroxybenzoate (amyl salicylate), armoise oil, benzyl 2-hydroxybenzoate (benzyl salicylate), bergamot oil, 4-tert-butyl-3-phenylpropanal (Bourgeonal), cedar leaf oil, 3,7-dimethyloct-6-en-1-ol (citronellol), (E)-1-(2,6,6-trimethyl-1-cyclohexenyl)but-2-en-1-one (beta damas-cene), 2-methyl-1-phenylpropan-2-yl acetate (dimethyl benzyl carbinyl acetate), ethyl 2,6,6-trimethylcyclohexadienecarboxylate (Ethyl Safranate), methyl 2,4-dihydroxy-3,6-dimethylbenzoate (Evernyl or Everniate), (E)-3,7-dimethylocta-2,6-dienenitrile (geranyl nitrile), 3-(1,3-benzodioxol-5-yl)-2-methylpropanal (Helional or Tropional), Z-hex-3-enol, hexyl 2-hydroxybenzoate (hexyl salicylate), lemon oil, 2,4-dimethyl-3-cyclohexene-1-carbaldehyde (Cyclal C or Tricyclal or Ligustral), 3-(4-(1,1-dimethylethyl)phenyl-2-methylpropanal (Lilial), 4-(4-hydroxy-4-methylpentyl) cyclohex-3-enecarbaldehyde (Lyral), 3-methyl-5-phenylpentanol (Mefrosol), orange oil, orange terpenes, tagetes oil, 3,7-dimethyloctan-1-ol (pelargol), and mixtures thereof,
(iv) the neutral fragrance materials N are selected from the group consisting of benzyl acetate, cassis base, 2-methyl-3-(4-(1-methylethyl)phenyl)propanal (cyclamen aldehyde), (5R)-2-methyl-5-prop-1-en-2-ylcyclohex-2-en-1-one (carvone), (E)-3-phenylprop-2-en-1-ol (cinnamic alcohol), 2-methoxy-4-propylphenol (dihydro-eugenol), 2,6-dimethyloct-7-en-2-ol (dihydromyrcenol), 4-allyl-2-methoxyphenol (eugenol), galbanum, 5-hexyloxolan-2-one (gamma decalactone), 7-hydroxy-3,7-dimethyloctanal (hydroxycitronellal), 1H-indole, (E)-2-methoxy-4-(prop-1-en-1-yl)phenol (isoeugenol), jasmin oil, 3-pentyltetrahydro-2H-4-pyranyl ethanoate (Jasmopyrane Forte), 3,7-dimethylocta-1,6-dien-3-ol (linalool), 3,7-dimethylocta-1,6-dien-3-yl acetate (linalyl acetate), methyl 3-oxo-2-pentylcyclopentaneacetate (methyl dihydrojasmonate), patchouli oil, 2-phenylethyl alcohol, 4-methyl-2-(2-methylprop-1-en-1-yl)tetrahydro-2H-pyran (rose oxide), rose oil, 5-(2,2,3-trimethyl-3-cyclopentyl-3-methylpentan-2-ol (Sandalore), 1-phenylethyl acetate (styrallyl acetate), ylang-ylang, and mixtures thereof, and
(v) the other fragrance materials M comprises fragrance materials not included in the above, excluding odourless or low-odour solvents or diluents used as vehicles for fragrance materials.

8. The fragrance composition according to claim 7, comprising at least about 35%, more preferably at least about 45%, by weight in total of relaxing fragrance materials R.

9. The fragrance composition according to claim 7, comprising at least eight relaxing fragrance materials R, more preferably at least ten relaxing fragrance materials R.

10. The fragrance composition according to claim 7, comprising relaxing fragrance materials R selected from one or more of the following groups:
(ii1) one or more musk ingredients selected from the group consisting of 1,4-dioxacycloheptadecane-5,17-dione (ethylene brassylate), 4,6,6,7,8,8-hexamethyl-1,3,4,6,7,8-hexahydrocyclopenta[g]isochromene (Galaxolide), mixtures of cyclohexa-decanolide and cyclopentadecanone (Silvanone), 1-(1,1,2,6-tetramethyl-3-propan-2-yl-2,3-dihydroinden-5-yl)ethanone (Traseolide), cyclohexadecanolide, cyclopenta-decanone, 17-oxacycloheptadec-6-en-1-one (ambrettolide), (9Z)-cycloheptadec-9-en-1-one (Civettone), (E)-3-methyl-5-cyclotetradecen-1-one (Cosmone), 6-acetyl-1,1,3,4,4,6-hexamethyltetrahydronaphthalene (Extralide), 1-(1,1,2,3,3,6-hexamethyl-2H-inden-5-yl)ethanone (Fixolide), (12E)-1-oxacyclohexadec-12-en-2-one (Habano-lide), [2-[1-(3,3-dimethylcyclohexyl)ethoxy]-2-methylpropyl]propanoate (Helvetolide), octahydrohexamethyl naphthoxirene (Moxalone), (5E)-3-methylcyclopentadec-5-en-1-one (Muscenone), 3-methyl-1-cyclopentadecanone (Muscone), 4-tert-butyl-2,6-dimethyl-3,5-dinitroacetophenone (musk ketone), 1,7-dioxacycloheptadecan-8-one (musk R1), (10Z)-13-methyl-1-oxacyclopentadec-10-en-2-one (Nirvanolide), 2-[1-(3,3-dimethylcyclohexyl)ethoxy]-2-methylpropyl cyclopropanecarboxylate (Sereno-lide), (3′E)-2-((3′,5′-dimethylhex-3′-en-2′-yl)oxy)-2-methylpropyl cyclopropanecarboxy-late (Sylkolide), 1,15-pentadecanolide (Thibetolide), (5Z)-cyclohexadec-5-en-1-one (Velvione), and mixtures thereof;
(ii2) one or more floral-orris ingredients selected from the group consisting of N-1-(2,6,6-trimethyl-1-cyclohex-2-enyl)pent-1-en-3-one (Isoraldeine), 4-(2,6,6-trimethyl-1-cyclo-hex-2-enyl)pentanal (Cetonal), (E)-1-(2,6,6-trimethylcyclohex-2-en-1-yl)hepta-1,6-dien-3-one (cetone V), 4-(2,6,6-trimethylcyclohex-1-en-1-yl)butan-2-one (dihydro-ionone beta), 7,9-dimethylspiro[5.5]undecan-3-one (Dispirone), (E)-4-(2,6,6-trimethyl-cyclohex-1-en-1-yl)but-3-en-2-one (ionone beta), (E)-4-(2,6,6-trimethylcyclohex-2-en-1-yl)but-3-en-2-one (irisone alpha), (E)-4-(2,5,6,6-tetramethylcyclohex-2-en-1-yl)but-3-en-2-one (irone alpha), (E)-3-methyl-4-(2,6,6-trimethylcyclohex-2-en-1-yl)but-3-en-2-one (Isoraldeine cetone alpha or alpha-iso-methyl ionone), (Z)-3,4,5,6,6-pentamethylhept-3-en-2-one (Koavone), and mixtures thereof,
(ii3) one or more sweet-vanilla ingredients selected from the group consisting of benzoin resinoids, 3-ethoxy-4-hydroxybenzaldehyde (ethyl vanillin), 4-formyl-2-methoxyphenyl 2-methylpropanoate (Isobutavan), 4-hydroxy-3-methoxybenzaldehyde (vanillin), 2-methoxy-4-methylphenol creosol, 2-ethoxy-4-(methoxymethyl)phenol (methyl diantilis), 2-propoxy-4-(methoxymethyl)phenol (propyl diantilis), 2-ethoxy-4-methylphenol (Ultravanil), and mixtures thereof;
(ii4) one or more sweet-heliotrope ingredients selected from the group consisting of 1-(4-methoxyphenyl)ethanone (acetanisole), 1-phenylethanone (acetophenone), 4-methoxybenzaldehyde (aubepine para cresol), benzo[d][1,3]dioxole-5-carbaldehyde (heliotropine), 1-(p-tolyl)ethanone methyl acetophenone, and mixtures thereof;
(ii5) one or more sweet-tonka ingredients selected from the group consisting of 2H-chromen-2-one (coumarin), octahydro-2H-chromen-2-one (bicyclononalactone), dec-9-en-1-yl (E)-3-(2-hydroxyphenyl)acrylate (Scentaurus Tonkarose), tonka bean oil, resinoid, extract or balsam, tonka roasted absolutes, coumarin replacers (e.g. Coumarex DB), and mixtures thereof; and/or
(ii6) one or more woody-vetiver ingredients selected from the group consisting of vetiver oil, (4,8-dimethyl-2-propan-2-ylidene-3,3a,4,5,6,8a-hexahydro-1H-azulen-6-yl) acetate (Vetiveryl Acetate), and mixtures thereof.

11. The fragrance composition according to claim 7, wherein the weight ratio of R to NR is at least 1:1, more preferably at least 2:1, and most preferably at least 3:1.

12. The fragrance composition according to claim 7, wherein the weight ratio of R to (M+NR) is at least 1:1, more preferably at least 2:1, and most preferably at least 3:1.

13. A consumer product comprising the fragrance composition according to claim 7.

14. A method of improving the relaxation state of a human subject, comprising the step of providing an effective amount of the fragrance composition according to claim 7 to the human subject.

15. A method of using a fragrance ingredient in improving the relaxation state of a human subject, wherein the fragrance ingredient is selected from the group consisting of 17-oxacycloheptadec-6-en-1-one (ambrettolide), (9Z)-cycloheptadec-9-en-1-one (Civettone), (E)-3-methyl-5-cyclotetradecen-1-one (Cosmone), 1-(1,1,2,3,3,6-hexamethyl-2H-inden-5-yl)ethanone (Fixolide), (12E)-1-oxacyclohexadec-12-en-2-one (Habanolide), [2-[1-(3,3-dimethylcyclohexyl)ethoxy]-2-methylpropyl]propanoate (Helvetolide), octahydrohexamethyl naphthoxirene (Moxalone), (5E)-3-methylcyclopentadec-5-en-1-one (Muscenone), 3-methyl-1-cyclopentadecanone (Muscone), 4-tert-butyl-2,6-dimethyl-3,5-dinitroacetophenone (musk ketone), 1,7-dioxacycloheptadecan-8-one (musk R1), (10Z)-13-methyl-1-oxacyclopentadec-10-en-2-one (Nirvanolide), 2-[1-(3,3-dimethylcyclohexyl)ethoxy]-2-methylpropyl cyclopropanecarboxylate (Serenolide), (3′E)-2-((3′,5′-dimethylhex-3′-en-2′-yl)oxy)-2-methylpropyl cyclopropanecarboxylate (Sylkolide), 1,15-pentadecanolide (Thibetolide), (5Z)-cyclohexadec-5-en-1-one (Velvione), N-1-(2,6,6-trimethyl-1-cyclohex-2-enyl)pent-1-en-3-one (Isoraldeine), 4-(2,6,6-trimethyl-1-cyclohex-2-enyl)pentanal (Cetonal), (E)-1-(2,6,6-trimethylcyclohex-2-en-1-yl)hepta-1,6-dien-3-one (cetone V), 4-(2,6,6-trimethylcyclohex-1-en-1-yl)butan-2-one (dihydro-ionone beta), 7,9-dimethylspiro[5.5]undecan-3-one (Dispirone), (E)-4-(2,6,6-trimethylcyclohex-1-en-1-yl)but-3-en-2-one (ionone beta), (E)-4-(2,6,6-trimethylcyclohex-2-en-1-yl)but-3-en-2-one (irisone alpha), (E)-4-(2,5,6,6-tetramethylcyclohex-2-en-1-yl)but-3-en-2-one (irone alpha), (E)-3-methyl-4-(2,6,6-trimethylcyclohex-2-en-1-yl)but-3-en-2-one (Isoraldeine cetone alpha), (Z)-3,4,5,6,6-pentamethylhept-3-en-2-one (Koavone), benzoin resinoids, 3-ethoxy-4-hydroxybenzaldehyde (ethyl vanillin), 4-formyl-2-methoxyphenyl 2-methylpropanoate (Isobutavan), 4-hydroxy-3-methoxybenzaldehyde (vanillin), 2-methoxy-4-methylphenol (creosol), 2-ethoxy-4-(methoxymethyl)phenol (methyl diantilis), 2-propoxy-4-(methoxymethyl)phenol (propyl diantilis), 1-(4-methoxyphenyl)ethanone (acetanisole), 1-phenylethanone (acetophenone), 4-methoxybenzaldehyde (aubepine para cresol), benzo[d][1,3]dioxole-5-carbaldehyde (heliotropine), 1-(p-tolyl)ethanone (methyl acetophenone), octahydro-2H-chromen-2-one (bicyclononalactone), dec-9-en-1-yl (E)-3-(2-hydroxyphenyl)acrylate (Scentaurus Tonkarose), tonka bean oil, resinoid, extract or balsam, tonka roasted absolutes, coumarin replacers (e.g. Coumarex DB), and mixtures thereof.
